SEQUENCE LISTING (27 sequences)

SEQ ID NO: 1 - DNA, 16 bp, Artificial Sequence
  Oligonucleotide containing DNA and RNA nucleotides
  modified_base (1)..(1): um
  modified_base (2)..(2): cm
  modified_base (3)..(3): cm
  modified_base (4)..(4): am
  modified_base (5)..(5): gm
  modified_base (6)..(6): gm
  modified_base (7)..(7): cm
  modified_base (8)..(8): gm
  modified_base (9)..(9): am
  modified_base (10)..(10): um
  modified_base (11)..(11): cm
  modified_base (16)..(16): cm
uccaggcgauctgtcc

SEQ ID NO: 2 - RNA, 140 nt, Artificial Sequence
  5′-UTR
ggacagaucgccuggagacgccauccacgcuguuuugaccuccauagaagacaccgggaccgauccagccuccgcggccgggaacggugcauuggaacgcggauuccccgugccaagagugacucaccguccuugacacg

SEQ ID NO: 3 - RNA, 140 nt, Artificial Sequence
  5′-UTR
agacagaucgccuggagacgccauccacgcuguuuugaccuccauagaagacaccgggaccgauccagccuccgcggccgggaacggugcauuggaacgcggauuccccgugccaagagugacucaccguccuugacacg

SEQ ID NO: 4 - RNA, 54 nt, Artificial Sequence
  5′-UTR
gagaauaaacuaguauucuucugguccccacagacucagagagaacccgccacc

SEQ ID NO: 5 - RNA, 47 nt, Artificial Sequence
  5′-UTR
gggaaauaagagagaaaagaagaguaagaagaaauauaagagccacc

SEQ ID NO: 6 - RNA, 14 nt, Artificial Sequence
  5′-UTR
ggagaaagcuuacc

SEQ ID NO: 7 - RNA, 57 nt, Artificial Sequence
  5′-UTR
gggaaauaagagagaaaagaagaguaagaagaaauauaagaccccggcgccgccacc

SEQ ID NO: 8 - RNA, 49 nt, Artificial Sequence
  5′-UTR
agggucaagauuagagaacggucguagcauuaucggagguucugccacc

SEQ ID NO: 9 - RNA, 48 nt, Artificial Sequence
  5′-UTR
gggucaagauuagagaacggucguagcauuaucggagguucugccacc

SEQ ID NO: 10 - DNA, 16 bp, Artificial Sequence
  Oligonucleotide containing DNA and RNA nucleotides
uccaggcgauctgtcc

SEQ ID NO: 11 - DNA, 16 bp, Artificial Sequence
  Oligonucleotide containing DNA and RNA nucleotides
uucucucuuauttccc

SEQ ID NO: 12 - DNA, 19 bp, Artificial Sequence
  Oligonucleotide containing DNA and RNA nucleotides
cuuuucucucuuauttccc

SEQ ID NO: 13 - DNA, 16 bp, Artificial Sequence
  Oligonucleotide containing DNA and RNA nucleotides
cagaagaauactagtu

SEQ ID NO: 14 - DNA, 20 bp, Artificial Sequence
  Oligonucleotide containing DNA and RNA nucleotides
ggaccagaagaauactagtu

SEQ ID NO: 15 - DNA, 20 bp, Artificial Sequence
  Oligonucleotide containing DNA and RNA nucleotides
cguucucuaaucuugacccu

SEQ ID NO: 16 - DNA, 16 bp, Artificial Sequence
  Oligonucleotide containing DNA and RNA nucleotides
cucuaaucuugacccu

SEQ ID NO: 17 - DNA, 20 bp, Artificial Sequence
  Oligonucleotide containing DNA and RNA nucleotides
ccguucucuaaucuugaccc

SEQ ID NO: 18 - DNA, 15 bp, Artificial Sequence
  Oligonucleotide containing DNA and RNA nucleotides
cucuaaucuugaccc

SEQ ID NO: 19 - DNA, 16 bp, Artificial Sequence
  Oligonucleotide containing DNA and RNA nucleotides
  modified_base (1)..(1): um
  modified_base (2)..(2): um
  modified_base (3)..(3): cm
  modified_base (4)..(4): um
  modified_base (5)..(5): cm
  modified_base (6)..(6): um
  modified_base (7)..(7): cm
  modified_base (8)..(8): um
  modified_base (9)..(9): um
  modified_base (10)..(10): am
  modified_base (11)..(11): um
  modified_base (16)..(16): cm
uucucucuuauttccc

SEQ ID NO: 20 - DNA, 19 bp, Artificial Sequence
  Oligonucleotide containing DNA and RNA nucleotides
  modified_base (1)..(1): cm
  modified_base (2)..(2): um
  modified_base (3)..(3): um
  modified_base (4)..(4): um
  modified_base (5)..(5): um
  modified_base (6)..(6): cm
  modified_base (7)..(7): um
  modified_base (8)..(8): cm
  modified_base (9)..(9): um
  modified_base (10)..(10): cm
  modified_base (11)..(11): um
  modified_base (12)..(12): um
  modified_base (13)..(13): am
  modified_base (14)..(14): um
  modified_base (19)..(19): cm
cuuuucucucuuauttccc

SEQ ID NO: 21 - DNA, 16 bp, Artificial Sequence
  Oligonucleotide containing DNA and RNA nucleotides
  modified_base (1)..(1): cm
  modified_base (2)..(2): am
  modified_base (3)..(3): gm
  modified_base (4)..(4): am
  modified_base (5)..(5): am
  modified_base (6)..(6): gm
  modified_base (7)..(7): am
  modified_base (8)..(8): am
  modified_base (9)..(9): um
  modified_base (10)..(10): am
  modified_base (11)..(11): cm
  modified_base (16)..(16): um
cagaagaauactagtu

SEQ ID NO: 22 - RNA, 47 nt, Artificial Sequence
  5′-UTR
aacuaguauucuucugguccccacagacucagagagaacccgccacc

SEQ ID NO: 23 - DNA, 20 bp, Artificial Sequence
  Oligonucleotide containing DNA and RNA nucleotides
  modified_base (1)..(1): gm
  modified_base (2)..(2): gm
  modified_base (3)..(3): am
  modified_base (4)..(4): cm
  modified_base (5)..(5): cm
  modified_base (6)..(6): am
  modified_base (7)..(7): gm
  modified_base (8)..(8): am
  modified_base (9)..(9): am
  modified_base (10)..(10): gm
  modified_base (11)..(11): am
  modified_base (12)..(12): am
  modified_base (13)..(13): um
  modified_base (14)..(14): am
  modified_base (15)..(15): cm
  modified_base (20)..(20): um
ggaccagaagaauactagtu

SEQ ID NO: 24 - DNA, 20 bp, Artificial Sequence
  Oligonucleotide containing DNA and RNA nucleotides
  modified_base (1)..(1): cm
  modified_base (2)..(2): gm
  modified_base (3)..(3): um
  modified_base (4)..(4): um
  modified_base (5)..(5): cm
  modified_base (6)..(6): um
  modified_base (7)..(7): cm
  modified_base (8)..(8): um
  modified_base (9)..(9): am
  modified_base (10)..(10): am
  modified_base (11)..(11): um
  modified_base (12)..(12): cm
  modified_base (13)..(13): um
  modified_base (14)..(14): um
  modified_base (15)..(15): gm
  modified_base (20)..(20): um
cguucucuaaucuugacccu

SEQ ID NO: 25 - DNA, 16 bp, Artificial Sequence
  Oligonucleotide containing DNA and RNA nucleotides
  modified_base (1)..(1): cm
  modified_base (2)..(2): um
  modified_base (3)..(3): cm
  modified_base (4)..(4): um
  modified_base (5)..(5): am
  modified_base (6)..(6): am
  modified_base (7)..(7): um
  modified_base (8)..(8): cm
  modified_base (9)..(9): um
  modified_base (10)..(10): um
  modified_base (11)..(11): gm
  modified_base (16)..(16): um
cucuaaucuugacccu

SEQ ID NO: 26 - DNA, 20 bp, Artificial Sequence
  Oligonucleotide containing DNA and RNA nucleotides
  modified_base (1)..(1): cm
  modified_base (2)..(2): cm
  modified_base (3)..(3): gm
  modified_base (4)..(4): um
  modified_base (5)..(5): um
  modified_base (6)..(6): cm
  modified_base (7)..(7): um
  modified_base (8)..(8): cm
  modified_base (9)..(9): um
  modified_base (10)..(10): am
  modified_base (11)..(11): am
  modified_base (12)..(12): um
  modified_base (13)..(13): cm
  modified_base (14)..(14): um
  modified_base (15)..(15): um
  modified_base (20)..(20): cm
ccguucucuaaucuugaccc

SEQ ID NO: 27 - DNA, 15 bp, Artificial Sequence
  Oligonucleotide containing DNA and RNA nucleotides
  modified_base (1)..(1): cm
  modified_base (2)..(2): um
  modified_base (3)..(3): cm
  modified_base (4)..(4): um
  modified_base (5)..(5): am
  modified_base (6)..(6): am
  modified_base (7)..(7): um
  modified_base (8)..(8): cm
  modified_base (9)..(9): um
  modified_base (10)..(10): um
  modified_base (15)..(15): cm
cucuaaucuugaccc

(19) United States
(12) Patent Application Publication
(10) Pub. No.: US 2024/0263217 A1
(43) Pub. Date: Aug. 8, 2024

(54) ASSAY FOR QUANTITATIVE ASSESSMENT OF MRNA CAPPING EFFICIENCY

(71) Applicant: TRANSLATE BIO, INC., Lexington, MA (US)

(72) Inventors: Frank DEROSA, Chelmsford, MA (US); Anusha DIAS, Medfield, MA (US); Xiaobo GU, Belmont, MA (US)

(21) Appl. No.: 18/565,849

(22) PCT Filed: Jun. 3, 2022
(86) PCT No.: PCT/US2022/032068

§ 371 Date: Nov. 30, 2023

Related U.S. Application Data
(60) Provisional application No. 63/197,106, filed on Jun. 4, 2021.

(51) Int. Cl. C12Q 1/6825; C12N 15/10

(52) U.S. Cl. CPC C12Q 1/6825; C12N 15/10

(57) ABSTRACT

The invention relates to a method of quantifying capping efficiency in a sample from an in vitro transcription reaction mixture comprising a plurality of mRNA transcript, characterized by a step of contacting the mRNA transcripts with an oligonucleotide complementary to a sequence of nucleotides in the 5′ untranslated region of the mRNA transcripts to form an mRNA:DNA hybrid between the oligonucleotide and the sequence of nucleotides of the mRNA transcripts in order to release the first five, six, or seven nucleotides of the mRNA transcripts using nuclease (e.g., RNAse H) digestion.

### CROSS-REFERENCE TO RELATED APPLICATIONS

This application claims benefit of, and priority to U.S. Provisional Patent Application Ser. No. 63/197,106 filed on Jun. 4, 2021, the contents of which are incorporated herein in its entirety.

### INCORPORATION-BY-REFERENCE OF SEQUENCE LISTING

The present specification makes reference to a Sequence Listing (submitted electronically as a text file named MRT-2241WO-Sequence Listing. The text file was generated on Jun. 1, 2022 and is 13,515 bytes in size. The entire contents of the sequence listing are herein incorporated by reference.

### BACKGROUND

Messenger RNA (“mRNA”) therapy is becoming an increasingly important approach for the treatment of a variety of diseases. mRNA therapy requires effective delivery of the mRNA to the patient and efficient production of the protein encoded by the mRNA within the patient's body.

mRNA used in therapy is typically produced by in vitro transcription (IVT). Only mRNAs with a methylated 5′ cap structure are efficiently translated in vivo, but mechanisms of generating capped mRNA by IVT are imperfect. Therefore, accurate characterization of the capping efficiency is particularly important for determining the quality of mRNA for therapeutic applications.

The IVT process can include a cap analog which is added co-transcriptionally. Alternatively, the 5′ cap structure can be added enzymatically post-transcription, after the IVT reaction has been completed.

During co-transcriptional capping, an anti-reverse cap analog (ARCA) is included in the IVT reaction mixture. The resulting mRNA transcript includes a methylated Cap 0 structure, which needs to be further methylated to form a Cap 1 structure. The Cap 1 structure is typically required for effective translation of the capped mRNA in vivo. Methylation can be done by including 2′-O-methyltransferase in the IVT reaction mixture. An mRNA sample from an IVT reaction mixture subjected to co-transcriptional capping can comprise mRNA transcripts comprising Cap 0 or Cap 1 at the 5′ end of the first nucleotide of the sequence of nucleotides, and/or mRNA transcripts lacking a cap structure. The mRNA transcript without a 5′ cap structure and with Cap 0 and Cap 1 structure, respectively, are shown in FIG. 1. If an ARCA cap analog has been used, the Cap 0 and Cap 1 structures may further comprise methylation of m⁷G at either the 2′ or 3′ OH group of the ribose ring.

Enzymatic post-transcriptional capping involves three synthetic steps. In a first step, a guanylyltransferase adds a guanine (Cap G) to the 5′ end of the in vitro transcribed mRNA using GTP as a substrate. In a second step, guanine methyltransferase adds a methyl group to form a Cap 0 structure. In a third step, 2′-O-methyltransferase adds a second methyl group to form a Cap 1 structure. This process is depicted in FIG. 2. An mRNA sample from an IVT reaction mixture subjected to enzymatic post-transcriptional capping can comprise mRNA transcripts comprising one of a plurality of different cap structures at the 5′ end of the first nucleotide of the sequence of nucleotides, and/or mRNA transcripts lacking a cap structure.

Methods of determining capping efficiency are known in the art. For instance, one method involves inclusion of [α-³²P]GTP in the IVT reaction mixture for which G is the first nucleotide of the sequence of nucleotides of the mRNA transcript. Following RNase T2 digestion, uncapped mRNA will release p3G[³²P] and its abundance relative to capped mRNA, for instance ^{m7}Gp3G[³²P], can be determined by analytical methods such as anion exchange chromatography. However, radiolabeling the sample prohibits use of the mRNA sample in the IVT reaction mixture in many downstream applications, and therefore radiolabeling methods are typically run in parallel as a separate control reaction. Such simultaneous but separate samples not only add to the number of samples that need to processed, but are inherently variable due to intra-operator error and minute variations in reaction conditions.

Radiolabel-free methods of measuring capping efficiency are also known. One such method is described in WO 2017/098468. It involves using an oligonucleotide probe complementary to the 5′ end of the mRNA. The probe comprises a tag that permits binding to a substrate such that the substrate can be used to enrich RNase H cleaved 5′ ends from a pool of synthesized mRNA molecules. However, the reagents required for the enrichment step (e.g., magnetic beads) add to the cost of the assay. Enrichment is also accompanied by an inherent reduction in yield, reducing the sample-efficiency of the assay.

Therefore, there is a need for cost-efficient, sample-efficient and time-efficient methods of quantifying capping efficiency in an mRNA sample from IVT reaction mixture.

### SUMMARY OF INVENTION

The present invention provides improved methods for accurately and quantitatively determining the capping efficiency of mRNA transcripts which have been synthesized by in vitro transcription (IVT). In particular, the invention relates to improved methods to quantify the capping efficiency of mRNA transcripts directly from an mRNA preparation sample (e.g., from an IVT reaction mixture) without pre-processing the mRNA preparation sample. Among other things, mRNA capping efficiency methods provided herein combine mRNA:DNA hybridization reaction, nuclease cleavage and MS and LC-MS (Mass Spectrometry/Liquid Chromatography-Mass Spectrometry) quantification in a one-step assay within a single reaction vessel. The improved method is a reliable, quick and efficient quantitative approach for assessing mRNA capping efficiency. The present invention is particularly useful for quality control during mRNA manufacture and for characterization of mRNA as an active pharmaceutical ingredient (API) in a final therapeutic product. The one-step simplicity using a single reaction vessel can also allow automation of the capping efficiency quantification.

The present invention is, in part, based on highly efficient and accurate enzymatic release of the first five, six or seven 5′ nucleotides of an mRNA transcript. This is achieved using an oligonucleotide to form an mRNA:DNA hybrid between the oligonucleotide and the second to fifth nucleotides, the third to sixth nucleotides, or the fourth to seventh nucleotides of the mRNA transcript, respectively to guide nuclease (e.g., RNAse H) activity such that the first five, six or seven 5′ nucleotides of mRNA are released from the remainder of the mRNA transcript. This is illustrated in FIG. 3, which depicts the use on an oligonucleotide to form an mRNA:DNA hybrid between the oligonucleotide and the second to fifth nucleotides to guide nuclease (e.g., RNAse H) activity such that the first five 5′ nucleotides of mRNA are released from the remainder of the mRNA transcript. By analyzing only the first five, six or seven 5′ nucleotides in the sequence of nucleotides of the mRNA transcripts, the invention provides a highly sensitive method for determining whether mRNA transcripts synthesized by IVT comprise a Cap 1 structure (e.g., as opposed to an incompletely methylated Cap 0 structure).

In particular, the present invention provides methods of quantifying mRNA capping efficiency in an mRNA sample from an IVT reaction mixture, comprising steps of: (a) providing the mRNA sample, wherein the mRNA sample contains a plurality of mRNA transcripts comprising a sequence of nucleotides, wherein a first portion of the mRNA transcripts comprises a Cap 1 structure at the 5′ end of the first nucleotide of the sequence of nucleotides; (b) contacting the mRNA sample with an oligonucleotide complementary to the mRNA transcripts to form an mRNA:DNA hybrid between the oligonucleotide and (i) the second to fifth nucleotides, (ii) the third to sixth nucleotides, or (iii) the fourth to seventh nucleotides of the sequence of nucleotides of the mRNA transcripts; (c) contacting the sample obtained in step (b) with nuclease (e.g., RNase H) to release (i) the first five 5′ nucleotides of the mRNA, (ii) the first six nucleotides, or (iii) the first seven nucleotides, respectively; and (d) analyzing the sample obtained in step (c) to determine the first portion of mRNA transcripts comprising the Cap 1 structure in the mRNA sample, wherein steps (a) to (c) proceed subsequent to one another in the same reaction vessel. The method of the present invention is a reliable, quick and efficient quantitative approach for assessing mRNA capping efficiency. The present invention is particularly useful for quality control during mRNA manufacture and for characterization of mRNA as an active pharmaceutical ingredient (API) in a final therapeutic product.

In some embodiments, the invention provides a method of quantifying mRNA capping efficiency in an mRNA sample from an IVT reaction mixture, comprising steps of: (a) providing the mRNA sample, wherein the mRNA sample contains a plurality of mRNA transcripts comprising a sequence of nucleotides, wherein a first portion of the mRNA transcripts comprises a Cap 1 structure at the 5′ end of the first nucleotide of the sequence of nucleotides; (b) contacting the mRNA sample with an oligonucleotide complementary to the mRNA transcripts to form an mRNA:DNA hybrid between the oligonucleotide and the second to fifth nucleotides of the sequence of nucleotides of the mRNA transcripts; (c) contacting the sample obtained in step (b) with nuclease (e.g., RNase H) to release the first five 5′ nucleotides of the mRNA; and (d) analyzing the sample obtained in step (c) to determine the first portion of mRNA transcripts comprising the Cap 1 structure in the mRNA sample, wherein steps (a) to (c) proceed subsequent to one another in the same reaction vessel.

In some embodiments, the invention provides a method of quantifying mRNA capping efficiency in an mRNA sample from an IVT reaction mixture, comprising steps of: (a) providing the mRNA sample, wherein the mRNA sample contains a plurality of mRNA transcripts comprising a sequence of nucleotides, wherein a first portion of the mRNA transcripts comprises a Cap 1 structure at the 5′ end of the first nucleotide of the sequence of nucleotides; (b) contacting the mRNA sample with an oligonucleotide complementary to the mRNA transcripts to form an mRNA:DNA hybrid between the oligonucleotide and the third to sixth nucleotides of the sequence of nucleotides of the mRNA transcripts; (c) contacting the sample obtained in step (b) with nuclease (e.g., RNase H) to release the first six 5 nucleotides of the mRNA; and (d) analyzing the sample obtained in step (c) to determine the first portion of mRNA transcripts comprising the Cap 1 structure in the mRNA sample, wherein steps (a) to (c) proceed subsequent to one another in the same reaction vessel.

In some embodiments, the invention provides a method of quantifying mRNA capping efficiency in an mRNA sample from an IVT reaction mixture, comprising steps of: (a) providing the mRNA sample, wherein the mRNA sample contains a plurality of mRNA transcripts comprising a sequence of nucleotides, wherein a first portion of the mRNA transcripts comprises a Cap 1 structure at the 5′ end of the first nucleotide of the sequence of nucleotides; (b) contacting the mRNA sample with an oligonucleotide complementary to the mRNA transcripts to form an mRNA:DNA hybrid between the oligonucleotide and the fourth to seventh nucleotides of the sequence of nucleotides of the mRNA transcripts; (c) contacting the sample obtained in step (b) with RNase H to release the first seven 5′ nucleotides of the mRNA; and (d) analyzing the sample obtained in step (c) to determine the first portion of mRNA transcripts comprising the Cap 1 structure in the mRNA sample, wherein steps (a) to (c) proceed subsequent to one another in the same reaction vessel.

In some embodiments, steps (b) to (d) are performed by an automated system.

In some embodiments, the analysis can further determine the methylation status of the cap. In some embodiments, a method according to the present invention further includes a step of determining the relative abundance of cap structures comprising different methylation statuses. In some embodiments, a method of the present invention can be used to determine the portion of mRNA transcripts comprising the Cap G structure. In some embodiments, a method of the present invention can be used to determine the portion of mRNA transcripts capped with Cap 0. In some embodiments, a method according to the present invention can be used to determine the portion of mRNA transcripts comprising a Cap 0 or Cap G structure further comprising methylation of m⁷G at either the 2′ or 3′ OH group of the ribose ring.

In some embodiments, the cap has been added enzymatically post-transcription. In some embodiments, the cap has been added to the mRNA co-transcriptionally.

A suitable oligonucleotide for use in step (b) is typically about 10-25 (e.g., 14-21) nucleotides in length. It comprises DNA nucleotides flanked on one or both sides by one or more RNA nucleotides (e.g., by 1, 2, 3, 4, 5, or more RNA nucleotides). In some embodiments, a suitable oligonucleotide is 16 nucleotides in length. In particular embodiments, a suitable oligonucleotide contains four DNA nucleotides flanked on one or both sides by one or more RNA nucleotides (e.g., by 1, 2, 3, 4, 5, or more RNA nucleotides).

In some embodiments, a suitable oligonucleotide oligonucleotide is represented by the following formula: 5′-[R]ₙ[D]₄[R]₁-3′, wherein each R is an RNA nucleotide, each D is a DNA nucleotide, wherein n is between 10 and 20, the oligonucleotide has a GC content of about 40% to about 60%, and the mRNA:DNA hybrid has a melting temperature between about 50° C. and about 60° C. In some embodiments, the melting temperature is between 52° C. and 58° C. In some embodiments, n is between 11 and 15. In particular embodiments, each of the RNA nucleotides comprises a 2′-O-methyl ribose. In a specific embodiment, the oligonucleotide is represented by the following formula: 5′-[R]₁₁[D]₄[R]₁-3′, wherein each R is an RNA nucleotide, each D is a DNA nucleotide.

An exemplary oligonucleotide that was found to be particularly suitable for use with the invention has the sequence: 5′-mUmCmCmAmGmGmCmGmAmUmCTGTCmC-3′ [SEQ ID NO: 1], wherein: mU represents uridine with a 2′-O-methyl ribose; mC represents cytidine with a 2′-O-methyl ribose; mA represents adenine with a 2′-O-methyl ribose; mG represents guanidine with a 2′-O-methyl ribose; T represents deoxythymidine; G represents deoxyguanidine and C represents deoxycytidine. This oligonucleotide can be used with mRNAs comprising the following 5′ untranslated region (5′ UTR): GGACAGAUCGCCUGGAGACGCCAUCCACGCUGUUUUGACCUCCAUAGAAGAC ACCGGGACCGAUCCAGCCUCCGCGGCCGGGAACGGUGCAUUGGAACGCGGAUU CCCCGUGCCAAGAGUGACUCACCGUCCUUGACACG [SEQ ID NO: 2]. The oligonucleotide binding site in the 5′ UTR is shown in bold. When contacted with mRNA transcripts comprising the 5′ UTR of SEQ ID NO: 2, an mRNA:DNA hybrid is formed between the oligonucleotide and the second to fifth nucleotides of the sequence of nucleotides of the mRNA transcripts. Upon contact of the mRNA:DNA hybrid with RNase H the first five 5′ nucleotides (and the cap nucleotide, if present) are released from the mRNA transcripts.

In some embodiments, the method requires an input of not more than 100 pmol of in vitro transcribed mRNA in the mRNA sample provided in step (a) of the method.

In some embodiments, the method requires a total assay volume of not more than 100 μl.

In some embodiments, steps (b) to (c) of the method are performed in 90 minutes or less.

In some embodiments, no purification steps are required between steps (a) to (d) of the method.

In some embodiments, step (d) of the method comprises HPLC, optionally UHPLC. In some embodiments, step (d) of the method comprises mass spectrometry. In some embodiments, step (d) of the method comprises Liquid Chromatography-Mass Spectrometry (LC-MS).

In some embodiments, the sample provided in step (a) comprises a second portion of mRNA transcripts comprising a Cap 0 structure. In some embodiments, the sample provided in step (a) comprises a third portion of mRNA transcripts comprising a Gap G structure. In some embodiments, the sample provided in step (a) comprises a fourth portion of mRNA transcripts that do not comprise a cap structure.

In some embodiments, the first portion of mRNA transcripts comprising the Cap 1 structure is at least 90% for the IVT reaction mixture to be processed further. In some embodiments, the second portion of mRNA transcripts comprising the Cap 0 structure is not more than 10% for the IVT reaction mixture to be processed further. In some embodiments, the third portion of mRNA transcripts comprising the Cap G structure is not more than 10% for the IVT reaction mixture to be processed further. In some embodiments, the fourth portion of mRNA transcripts that do not comprise a cap structure is not more than 10% for the IVT reaction mixture to be processed further. In some embodiments, the further processing includes purification and/or encapsulation of the mRNA transcripts from the IVT reaction mixture. In some embodiments, the further processing includes use of the mRNA transcripts to manufacture a therapeutic composition.

In some embodiments, the invention relates to a method of manufacturing a therapeutic mRNA, wherein said method comprises (i) synthesizing the therapeutic mRNA using an in vitro transcription (IVT) reaction mixture; (ii) analyzing an mRNA sample from the IVT reaction mixture using a method of quantifying mRNA capping efficiency in accordance with the invention; and (iii) further processing the mRNA, if the first portion of mRNA transcripts comprising the Cap 1 structure in the mRNA is at least 90%. In some embodiments, further processing comprises purification of the therapeutic mRNA synthesized in step (i). In addition or as an alternative, further processing may comprise formulating the therapeutic mRNA synthesized in step (i). Formulation may comprise encapsulating the mRNA in a lipid nanoparticle.

Among other things, the present invention further provides compositions and kits for performing the inventive methods described herein. In some embodiments, the present invention provides a kit for quantifying mRNA capping efficiency, comprising one or more of: (1) a suitable oligonucleotide complementary to a sequence in the 5′ untranslated region of an mRNA transcript in a sample for which capping efficiency is to be quantified: (2) one or more reagents for annealing between DNA and RNA; (3) RNAse H; (4) one or more reagents for performing an analysis (e.g., a chromatographic column) and (5) one or more control samples.

In some embodiments, the oligonucleotide is capable of forming an mRNA:DNA hybrid between the oligonucleotide and the second to fifth nucleotides of the mRNA transcript, and each of the control samples comprises (i) a sequence of five nucleotides comprising a 5′ cap structure, and (ii) a sequence of five nucleotides lacking a cap structure or comprising a different cap structure to that in (i), at defined ratios to provide a reference. For example, a control sample may contain (i) a sequence of five nucleotides comprising a 5′ Cap 1 structure, and (ii) a sequence of five nucleotides lacking a cap structure at a ratio of 9:1. The control samples are adapted accordingly for oligonucleotides that are capable of forming an mRNA:DNA hybrid between the oligonucleotide and the third to sixth nucleotides, or the fourth to seventh nucleotides, respectively, of the sequence of nucleotides of the mRNA transcript, i.e., they comprise a sequence of six or seven nucleotides, respectively.

In some embodiments, one or more control samples according to (5) are used in a calibration step as part of the analysis according to step (d) of the method of the invention. In some embodiments, the one or more control samples are run in parallel to the analysis of the sample obtained according to step (c) of the method. In some embodiments, the control samples are modified, for example deuterated or radiolabeled, such that the sample obtained according to step (c) of the method can be spiked with one or more control samples for simultaneous measurement of the sample obtained according to step (c) of the method and the one or more control samples.

In some embodiments, a control sample according to (5) contains a sequence of five, six, or seven nucleotides, as appropriate, of which 60 to 100% comprise a Cap 1 structure, for example 70 to 98% Cap 1, or 80 to 95% Cap 1, or 90% Cap 1. In some embodiments, a control sample according to (5) contains a sequence of five, six, or seven nucleotides, as appropriate, of which 1 to 30% comprise a Cap G structure, for example 2 to 20% Cap G, or 3 to 15% Cap G, or 10% Cap G. In some embodiments, the control sample according to (5) contains a sequence of five, six, or seven nucleotides, as appropriate, of which 1 to 30% comprise a Cap 0 structure, for example 2 to 20% Cap 0, or 3 to 15% Cap 0, or 10% Cap 0. In some embodiments, a control sample according to (5) contains a sequence of five, six, or seven nucleotides, as appropriate, of which 1 to 30% do not comprise a cap structure, for example 2 to 20% lacking a cap structure, or 3 to 15% lacking a cap structure, or 10% lacking a cap structure.

In some embodiments, the invention provides a method of quantifying mRNA capping efficiency in an mRNA preparation sample, comprising steps of: (a) contacting the mRNA preparation sample with an oligonucleotide complementary to the mRNA transcripts to form an mRNA:DNA hybrid between the oligonucleotide and the second to fifth nucleotides, the third to sixth nucleotides, or the fourth to seventh nucleotides of the sequence of nucleotides of the mRNA transcripts; (b) contacting the sample obtained in step (b) with nuclease (e.g., RNase H) to release (i) the first five 5′ nucleotides of the mRNA, (ii) the first six nucleotides, or (iii) the first seven nucleotides, respectively; and (c) analyzing the released nucleotides in the sample to determine the relative amount of mRNA transcripts comprising the Cap 1 structure in the mRNA sample, wherein steps (a) to (c) proceed subsequent to one another in the same reaction vessel.

In some embodiments, the present invention provide a composition comprising purified in vitro synthesized mRNA molecules comprising the Cap 1 structure. In some embodiments, the composition of the present invention comprise at least 80% of mRNA molecules comprising the Cap 1 structure. In some embodiments, the composition of the present invention comprise at least 90% of mRNA molecules comprising the Cap 1 structure. In some embodiments, the composition of the present invention comprise at least 95% of mRNA molecules comprising the Cap 1 structure. In some embodiments, the composition of the present invention comprise more than 95% of mRNA molecules comprising the Cap 1 structure.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description

### BRIEF DESCRIPTION OF THE DRAWINGS

The drawings are for illustration purposes and are in no way limiting.

FIG. 1 shows schematically an mRNA transcript without a 5′ cap structure (A), an mRNA transcript with Cap 0 (B), and an mRNA transcript with a Cap 1 structure (C). The Cap 0 and Cap 1 structures may further comprise methylation of m⁷G at either the 2′ or 3′ ′OH group of the ribose ring (not shown).

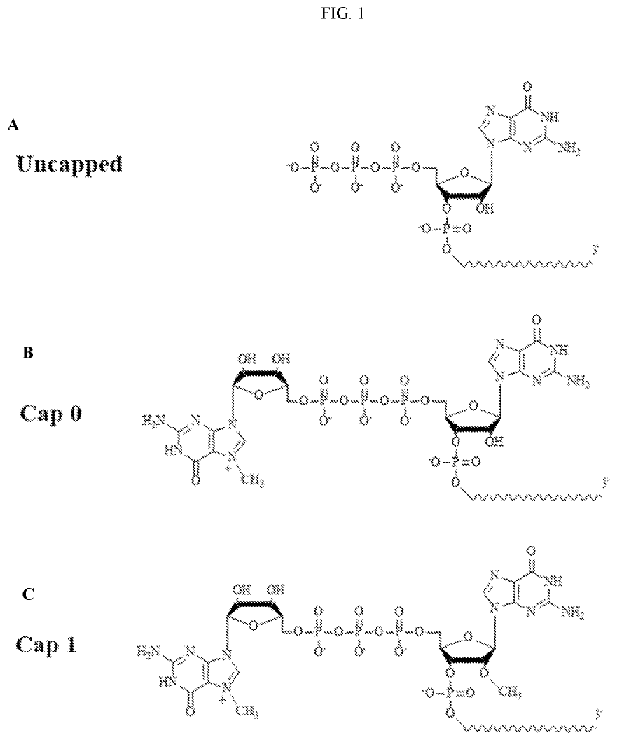

FIG. 2 illustrates schematically an enzymatic capping process.

FIG. 3 illustrates schematically how an oligonucleotide can be used to form an mRNA:DNA hybrid between the oligonucleotide and the second to fifth nucleotides of the mRNA transcript to guide RNAse H activity such that the first five 5′ nucleotides of mRNA are released from the remainder of the mRNA transcript. The dotted aspect of the oligonucleotide represents DNA nucleotides; the solid aspects represent RNA nucleotides.

FIG. 4 depicts an exemplary method for the analysis of the 5′ cap structure of a sample of in vitro transcribed mRNA transcripts. FIG. 4A shows the mRNA transcript with a hybridized oligonucleotide; the oligonucleotide DNA nucleotides are indicated by letters and the RNA nucleotides are represented by solid lines. The arrow indicates the RNase H digestion site. FIGS. 4B and 4C show UHPLC chromatograms of cleaved nucleotides resulting from the RNase H digestion.

### DEFINITIONS

In order for the present invention to be more readily understood, certain terms are first defined. Additional definitions for the following terms and other terms are set forth throughout the specification.

About: As used in this application, the terms “about” and “approximately” are used as equivalents. Any numerals used in this application with or without about/approximately are meant to cover any normal fluctuations appreciated by one of ordinary skill in the relevant art. Typically, the term “approximately” or “about” refers to a range of values that is within 10% (e.g., within 5%), or more typically 1%, of the stated reference value.

Affinity: As is known in the art, “affinity” is a measure of the tightness with which a particular ligand binds to (e.g., associates non-covalently with) and/or the rate or frequency with which it dissociates from, its partner. As is known in the art, any of a variety of technologies can be utilized to determine affinity. In many embodiments, affinity represents a measure of specific binding.

Anneal or hybridization: As used herein, the terms “anneal,” “hybridization,” and grammatical equivalent, refers to the formation of complexes (also called duplexes or hybrids) between nucleotide sequences which are sufficiently complementary to form complexes via Watson-Crick base pairing or non-canonical base pairing. It will be appreciated that annealing or hybridizing sequences need not have perfect complementary to provide stable hybrids. In many situations, stable hybrids will form where fewer than about 10% of the bases are mismatches. Accordingly, as used herein, the term “complementary.” refers to a nucleic acid molecule that forms a stable duplex with its complement under particular conditions, generally where there is about 90% or greater homology (e.g., about 95% or greater, about 98% or greater, or about 99% or greater homology). Those skilled in the art understand how to estimate and adjust the stringency of hybridization conditions such that sequences that have at least a desired level of complementarity will stably hybridize, while those having lower complementarity will not. For examples of hybridization conditions and parameters, see, for example, Sambrook et al., “Molecular Cloning: A Laboratory Manual”, 1989, Second Edition, Cold Spring Harbor Press: Plainview, N Y and Ausubel, “Current Protocols in Molecular Biology”, 1994, John Wiley & Sons: Secaucus, NJ. Complementarity between two nucleic acid molecules is said to be “complete”, “total” or “perfect” if all the nucleic acid's bases are matched, and is said to be “partial” otherwise.

Cap 1: As used herein, an mRNA transcript comprising a Cap 1 structure refers to an RNA transcript comprising a cap structure in which at least both the N7 amine of the guanine cap is methylated and the first nucleotide in the sequence of nucleotides of the mRNA transcript is methylated at the 2′OH of the ribose. An mRNA transcript comprising a Cap 1 structure may comprise further modifications. For example, the 2′ or 3′ OH group of ‘the cap ribose may be methylated. As another example, the 2′OH of the second nucleotide in the sequence of nucleotides of the mRNA transcript may be methylated.

Cap 0: As used herein, an mRNA transcript comprising a Cap 0 structure refers to an mRNA transcript comprising a cap structure in which the N7 amine of the guanine cap is methylated but the first nucleotide in the sequence of nucleotides of the mRNA transcript is not methylated at the 2′OH of the ribose. An mRNA transcript comprising a Cap 0 structure may comprise further modifications. For example, the 2′ or 3′ OH group of the cap ribose may be methylated.

Chromatography: As used herein, the term “chromatography” refers to a technique for separation of mixtures. Typically, the mixture is dissolved in a fluid called the “mobile phase,” which carries it through a structure holding another material called the “stationary phase.” Column chromatography is a separation technique in which the stationary bed is within a tube, i.e., a column.

Control: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

Kit: As used herein, the term “kit” refers to any delivery system for delivering materials. Such delivery systems may include systems that allow for the storage, transport, or delivery of various diagnostic or therapeutic reagents (e.g, oligonucleotides, antibodies, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components in a single container (e.g., in a single box housing each of the desired components). The term “kit” includes both fragmented and combined kits.

Nucleoside: The term “nucleoside” or “nucleobase”, as used herein, refers to adenine (“A”), guanine (“G”), cytosine (“C”), uracil (“U”), thymine (“T”) and analogs thereof linked to a carbohydrate, for example D-ribose (in RNA) or 2′-deoxy-D-ribose (in DNA), through an N-glycosidic bond between the anomeric carbon of the carbohydrate (1′-carbon atom of the carbohydrate) and the nucleobase. When the nucleobase is purine, e.g., A or G, the ribose sugar is generally attached to the N9-position of the heterocyclic ring of the purine. When the nucleobase is pyrimidine, e.g., C, T or U, the sugar is generally attached to the NI-position of the heterocyclic ring. The carbohydrate may be substituted or unsubstituted. Substituted ribose sugars include, but are not limited to, those in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Ribose examples include ribose, 2′-deoxyribose, 2′,3′-dideoxyribose, 2′-haloribose, 2′-fluororibose, 2′-chlororibose, and 2′-alkylribose, e.g., 2′-O-methyl, 4′-alpha-anomeric nucleotides, l′-alpha-anomeric nucleotides (Asseline et al., NUCL. ACIDS RES., 19:4067-74 [1991]), 2′-4′- and 3′-4′-linked and other “locked” or “LNA,” bicyclic sugar modifications (WO 98/22489; WO 98/39352; WO 99/14226).

Nucleotide: The term “nucleotide” as used herein means a nucleoside in a phosphorylated form (a phosphate ester of a nucleoside), as a monomer unit or within a polynucleotide polymer. “Nucleotide 5′-triphosphate” refers to a nucleotide with a triphosphate ester group at the 5′ position, sometimes denoted as “NTP”, or “dNTP” and “ddNTP” to particularly point out the structural features of the ribose sugar. The triphosphate ester group may include sulfur substitutions for the various oxygen moieties, e.g., alpha-thio-nucleotide 5′-triphosphates. Nucleotides can exist in the mono-, di-, or tri-phosphorylated forms. The carbon atoms of the ribose present in nucleotides are designated with a prime character (′) to distinguish them from the backbone numbering in the bases. For a review of polynucleotide and nucleic acid chemistry see Shabarova, Z. and Bogdanov, A. Advanced Organic Chemistry of Nucleic Acids, VCH, New York, 1994.

Nucleic acid: The terms “nucleic acid”, “nucleic acid molecule”, “polynucleotide” or “oligonucleotide” may be used herein interchangeably. They refer to polymers of nucleotide monomers or analogs thereof, such as deoxyribonucleic acid (DNA) and ribonucleic acid (RNA) and combinations thereof. The nucleotides may be genomic, synthetic or semi-synthetic in origin. Unless otherwise stated, the terms encompass nucleic acid-like structures with synthetic backbones, as well as amplification products. As will be appreciated by one skilled in the art, the length of these polymers (i.e., the number of nucleotides it contains) can vary widely, often depending on their intended function or use. Polynucleotides can be linear, branched linear, or circular molecules. Polynucleotides also have associated counter ions, such as H⁺, NH₄⁺, trialkylammonium, Mg⁺, Na⁺ and the like. A polynucleotide may be composed entirely of deoxyribonucleotides, entirely of ribonucleotides, or chimeric mixtures thereof. Polynucleotides may be composed of internucleotide nucleobase and sugar analogs.

Oligonucleotide: In some embodiments, the term “oligonucleotide” is used herein to denote a polynucleotide that comprises between about 5 and about 150 nucleotides, e.g., between about 10 and about 100 nucleotides, between about 15 and about 75 nucleotides, or between about 15 and about 50 nucleotides. In the context of the invention, an oligonucleotide typically comprises about 10-25 nucleotides (e.g., 14-21 nucleotides). Throughout the specification, whenever an oligonucleotide is represented by a sequence of letters (chosen, for example, from the four base letters: A, C, G, and T, which denote adenosine, cytidine, guanosine, and thymidine, respectively), the nucleotides are presented in the 5′ to 3′ order from the left to the right. A “polynucleotide sequence” refers to the sequence of nucleotide monomers along the polymer. Unless denoted otherwise, whenever a polynucleotide sequence is represented, it will be understood that the nucleotides are in 5′ to 3′ orientation from left to right.

Nucleotide analogs: Nucleic acids, polynucleotides and oligonucleotides may be comprised of standard nucleotide bases or substituted with nucleotide isoform analogs, including, but not limited to iso-C and iso-G bases, which may hybridize more or less permissibly than standard bases, and which will preferentially hybridize with complementary isoform analog bases. Many such isoform bases are described, for example, by Benner et al., (1987) Cold Spring Harb. Symp. Quant. Biol. 52, 53-63. Analogs of naturally occurring nucleotide monomers include, for example, 7-deazaadenine, 7-deazaguanine, 7-deaza-8-azaguanine, 7-deaza-8-azaadenine, 7-methylguanine, inosine, nebularine, nitropyrrole (Bergstrom, J. Amer. Chem. Soc., 117:1201-1209 [1995]), nitroindole, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine (Seela, U.S. Pat. No. 6,147,199), 7-deazaguanine (Seela, U.S. Pat. No. 5,990,303), 2-azapurine (Seela, WO 01/16149), 2-thiopyrimidine, 6-thioguanine, 4-thiothymine, 4-thiouracil, 0-6-methylguanine, N-6-methyladenine, O-4-methylthymine, 5,6-dihydrothymine, 5,6-dihydrouracil, 4-methylindole, pyrazolo[3,4-D]pyrimidines, “PPG” (Meyer, U.S. Pat. Nos. 6,143,877 and 6,127,121: Gall, WO 01/38584), and ethenoadenine (Fasman (1989) in Practical Handbook of Biochemistry and Molecular Biology, pp. 385-394, CRC Press, Boca Raton, Fla.).

5′ terminus 3′ terminus: The terms “3′ end” and “3′ terminus”, as used herein in reference to a nucleic acid molecule, refer to the end of the nucleic acid which contains a free hydroxyl group attached to the 3′ carbon of the terminal pentose sugar. The term “5′ end” and “5′ terminus”, as used herein in reference to a nucleic acid molecule, refers to the end of the nucleic acid molecule which contains a free hydroxyl or phosphate group attached to the 5′ carbon of the terminal pentose sugar. When referring to nucleotide numbers in an mRNA transcript, unless stated otherwise, the cap nucleotide is not counted. For example, the first 5′ nucleotide refers to the 5′-most nucleotide containing a free hydroxyl or phosphate group attached to the 5′ carbon of the terminal pentose sugar in an uncapped mRNA transcript, or to the 5′-most nucleotide containing a cap structure attached to the 5′ carbon of the terminal pentose sugar in a capped mRNA transcript.

### DETAILED DESCRIPTION

The present invention provides improved methods of quantifying capping efficiency in an mRNA sample from an in vitro transcription (IVT) reaction mixture to determine the portion of mRNA transcripts comprising the Cap 1 structure.

The method is based on enzymatically releasing the first five, six or seven nucleotides of the sequence of nucleotides of an mRNA transcript. The released first five, six or seven nucleotides are generated by contacting an mRNA sample with an oligonucleotide complementary to the mRNA transcripts to form an mRNA:DNA hybrid between the oligonucleotide and (i) the second to fifth nucleotides, (ii) the third to sixth nucleotides, or (iii) the fourth to seventh nucleotides, respectively, of the sequence of nucleotides of the mRNA transcripts and contacting the sample with nuclease (e.g. RNase H). The sample is analyzed to determine the portion of mRNA transcripts comprising the Cap 1 structure in the mRNA sample.

mRNA transcripts comprising a Cap 1 structure at the 5′ end of the first nucleotide of the sequence of nucleotides are efficiently translated. In contrast, mRNA transcripts comprising a Cap G or Cap 0 cap structure, or mRNA transcripts lacking a cap structure, will not be translated as efficiently as mRNA transcripts comprising Cap 1. Whether capping of an mRNA is carried out co-transcriptionally or enzymatically post-transcription, at least a portion of mRNA transcripts will not comprise a Cap 1 structure. It is therefore important that the capping efficiency in an mRNA sample from an IVT reaction mixture be determined prior to downstream use. For example, the mRNA sample from an IVT reaction mixture may also comprise mRNA transcripts comprising a Cap 0 structure. In some embodiments, the mRNA sample from an IVT reaction mixture also comprises a portion of mRNA transcripts comprising a Cap G structure. In some embodiments, the mRNA sample from an IVT reaction mixture also comprises a portion of mRNA transcripts that do not comprise a cap structure.

Accordingly, in some embodiments, the method of the invention determines the relative abundance of mRNA transcripts comprising Cap 1 to mRNA transcripts comprising a different cap (e.g., Cap 0 or Cap G). In some embodiments, the analysis can determine the relative abundance of mRNA transcripts comprising the Cap 0 structure. In some embodiments, the analysis can determine the relative abundance of mRNA transcripts comprising the Cap G structure. In some embodiments, the analysis can determine the relative abundance of mRNA transcripts lacking a cap structure.

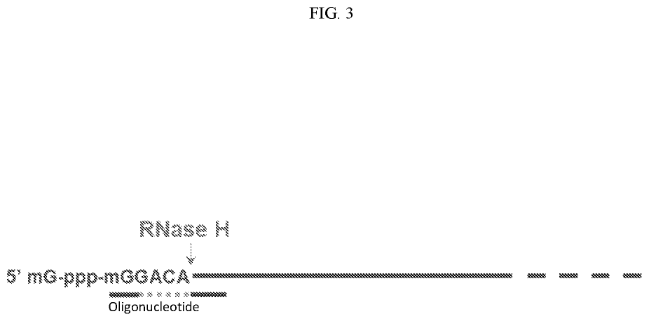

The inventors found that enzymatic release of the first five 5′ nucleotides of an mRNA transcript is particular useful in determining the relative abundance of mRNA transcripts comprising Cap 1 to mRNA transcripts comprising Cap 0 or Cap G. This is achieved using an oligonucleotide to form an mRNA:DNA hybrid between the oligonucleotide and the second to fifth nucleotides of the mRNA transcript, as depicted in FIG. 3, to guide nuclease (e.g., RNAse H) activity such that the first five 5′ nucleotides of mRNA are released from the remainder of the mRNA transcript. By analyzing only the first five 5′ nucleotides in the sequence of nucleotides of the mRNA transcripts, the inventors were able to provide a highly sensitive method for determining whether mRNA transcripts synthesized by IVT comprise a Cap 1 structure (e.g., as opposed to an incompletely methylated Cap 0 structure).

In certain embodiments, the method of the invention determines a methylation profile of the mRNA transcripts in an mRNA sample from an IVT reaction mixture.

The method of the invention can quickly, reliably and efficiently determine the capping efficiency and assess whether the majority of mRNA transcripts in an mRNA sample from an IVT reaction mixture comprises a Cap 1 structure and therefore are suitable to efficiently express the mRNA-encoded protein, e.g., in the context of a therapeutic application, such as vaccination with an mRNA encoding an antigen, or a treatment of a protein deficiency, wherein the mRNA encodes a protein deficient in a subject, e.g. due to a functional deficiency or absence of the protein as a result of a genetic mutation.

mRNA Capping

Typically, eukaryotic mRNAs bear a “cap” structure at their 5′-termini, which plays an important role in translation. For example, the cap plays a pivotal role in mRNA metabolism, and is required to varying degrees for processing and maturation of an RNA transcript in the nucleus, transport of mRNA from the nucleus to the cytoplasm, mRNA stability, and efficient translation of the mRNA to protein. The 5′ cap structure is involved in the initiation of protein synthesis of eukaryotic cellular and eukaryotic viral mRNAs and in mRNA processing and stability in vivo (see, e.g, Shatkin, A. J., CELL, 9: 645-653 (1976); Furuichi, et al., NATURE, 266: 235 (1977); FEDERATION OF EXPERIMENTAL BIOLOGISTS SOCIETY LETTER 96: 1-11 (1978): Sonenberg, N., PROG. NUC. ACID RES MOL BIOL, 35: 173-207 (1988)). Components of the machinery required for initiation of translation of an mRNA include specific cap-binding proteins (see, e.g., Shatkin, A. J., CELL, 40: 223-24 (1985); Sonenberg, N., PROG. NUC. ACID RES MOL BIOL, 35: 173-207 (1988)). The cap of mRNA is recognized by the translational initiation factor eIF4E (Gingras, et al., ANN. REV. BIOCHEM. 68: 913-963 (1999): Rhoads, R. E., J. BIOL. CHEM. 274: 30337-3040 (1999)). The 5′ cap structure also provides resistance to 5′-exonuclease activity and its absence results in rapid degradation of the mRNA (see, e.g., Ross, J., MOL. BIOL. MED. 5: 1-14 (1988): Green, M. R. et al., CELL, 32: 681-694 (1983)). Since the primary transcripts of many eukaryotic cellular genes and eukaryotic viral genes require processing to remove intervening sequences (introns) within the coding regions of these transcripts, the benefit of the cap also extends to stabilization of such pre-mRNA.

Capped RNAs have been reported to be translated more efficiently than uncapped transcripts in a variety of in vitro translation systems, such as rabbit reticulocyte lysate or wheat germ translation systems (see, e.g., Shimotohno, K., et al., PROC. NATL. ACAD. SCI. USA, 74: 2734-2738 (1977): Paterson and Rosenberg, NATURE, 279: 692 (1979)). This effect is also believed to be due in part to protection of the RNA from exoribonucleases which may be present in the in vitro translation system, as well as other factors.

### Cap 1

Naturally-occurring cap structures comprise a 7-methyl guanosine that is linked via a triphosphate bridge to the 5′-end of the first nucleotide in the sequence of nucleotides of the mRNA transcript, resulting in m⁷G(5′)ppp(5′)N, where N is any nucleoside. In vivo, the cap is added enzymatically. The cap is added in the nucleus and is catalyzed by the enzyme guanylyl transferase. The addition of the cap to the 5′ terminal end of RNA occurs immediately after initiation of transcription. The cap nucleoside is in the reverse orientation to all the other nucleotides, i.e., m⁷G(5′)ppp(5′)Np. A cap structure comprising an N7 methyl guanosine and lacking 2′O methylation of the ribose of the first nucleotide in the sequence of nucleotides of the mRNA transcript is known as Cap 0. 2′O methylation of the ribose of the first nucleotide in the sequence of nucleotides of the mRNA transcript leads to m⁷G(5)ppp(5′)Nm, i.e. Cap 1. This 2′O methylation is important for the differentiation between ‘self’ and ‘non-self’ RNA, and is therefore important for increasing the translation efficiency of mRNA transcripts (see, e.g., Sikorski., et al., NUC. ACID RES, 48: 4 (2020)). One reported mechanism for the increased translation efficiency of mRNA transcripts comprising Cap 1 is that ribose 2′-O methylation of the first nucleotide of the sequence of nucleotides prevents recognition of the mRNA transcript by IFIT1 (IFN-induced protein with tetratricopeptide repeats-1), an inhibitor of translation (Abbas., et al., PNAS, 114: 11 (2017), Habjan., et al., PLOS PATHOG, 9: 10 (2013)).

Production of Capped mRNAs

Transcription of RNA usually starts with a nucleoside triphosphate (usually a purine, A or G). In vitro transcription typically comprises a phage RNA polymerase such as T7, T3 or SP6, a DNA template containing a phage polymerase promoter, nucleotides (ATP, GTP, CTP and UTP) and a buffer containing magnesium salt.

### Co-Transcriptional Capping

Co-transcriptional capping can be carried out with a pre-formed dinucleotide of the form m⁷G(5′)ppp(5)G (“m⁷GpppG”) as an initiator of transcription. Excess cap (e.g. m⁷GpppG) to GTP (4:1) increases the opportunity that each transcript will have a 5′ cap. Altering this ratio can affect the balance between achieving a high yield of transcription and minimizing the portion of mRNA transcripts lacking a cap structure. Kits for this type of capping of IVT mRNAs are commercially available, including the mMESSAGE mMACHINE® kit (Invitrogen). These kits will typically yield 80% capped RNA to 20% uncapped RNA, although total RNA yields are lower as GTP concentration becomes rate limiting as GTP is needed for the elongation of the transcript. A disadvantage of using m⁷G(5′)ppp(5)G, a pseudosymmetrical dinucleotide, is the propensity of the 3′-OH of either the G or m⁷G moiety to serve as the initiating nucleophile for transcriptional elongation. In other words, the presence of a 3′-OH on both the m⁷G and G moieties leads to up to half of the mRNAs incorporating caps in an improper orientation. This leads to the synthesis of two isomeric RNAs of the form m⁷G(5′)pppG(pN)ₙ and G(5)pppm⁷G(pN)n, in approximately equal proportions, depending upon the ionic conditions of the transcription reaction. Variations in the isomeric forms can adversely effect in vitro translation and are undesirable for a homogenous therapeutic product.

To prevent this reverse cap orientation, the usual form of a synthetic dinucleotide cap used in in vitro translation experiments is the Anti-Reverse Cap Analog (“ARCA”), which is generally a modified cap analog in which the 2′ or 3′ OH group is replaced with —OCH₃. Chemical modification of m⁷G at either the 2′ or 3′ OH group of the ribose ring results in the cap being incorporated solely in the forward orientation, even though the 2′ OH group does not participate in the phosphodiester bond (Jemielity, J. et al., “Novel ‘anti-reverse’ cap analogs with superior translational properties”, RNA, 9: 1108-1122 (2003)). The selective procedure for methylation of guanosine at N7 and 3′ O-methylation and 5′ diphosphate synthesis has been established (Kore, A. and Parmar, G. NUCLEOSIDES, NUCLEOTIDES, AND NUCLEIC ACIDS, 25: 337-340, (2006) and Kore, A. R., et al. NUCLEOSIDES, NUCLEOTIDES, AND NUCLEIC ACIDS 25(3): 307-14, (2006). Kits for capping of IVT mRNAs using ARCA caps are commercially available, including the mMESSAGE mMACHINE® T7 ULTRA kit (Invitrogen). Such kits typically recommend a ratio of cap analog to GTP of 4:1. Decreasing the ratio of cap analog to GTP typically increases the yield of the transcription reaction but decreases the portion of mRNA transcripts comprising a cap structure.

### Post-Transcriptional Capping

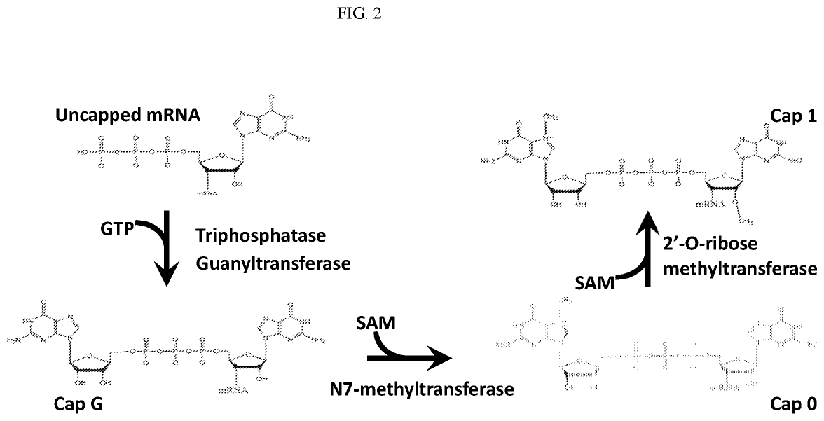

mRNA can also be capped post-transcriptionally in a three-step enzymatic process, outlined in FIG. 2. In a first step, a guanylyltransferase adds a guanine to the 5′ end of the first nucleotide of the sequence of nucleotides of the mRNA transcript using GTP as a substrate to form a Cap G structure. In a second step, N7 methyltransferase adds a methyl group to N7 of the cap guanine to form a Cap 0 structure. In a third step, 2′-O-methyltransferase adds a methyl group to the 2′ OH of the ribose of the first nucleotide in the sequence of nucleotides in the mRNA transcript to form a Cap 1 structure.

Methods of Quantifying mRNA Capping Efficiency

In some embodiments, the invention provides a method of quantifying mRNA capping efficiency in an mRNA sample from an IVT reaction mixture, comprising steps of: (a) providing the mRNA sample, wherein the mRNA sample contains a plurality of mRNA transcripts comprising a sequence of nucleotides, wherein a first portion of the mRNA transcripts comprises a Cap 1 structure at the 5′ end of the first nucleotide of the sequence of nucleotides: (b) contacting the mRNA sample with an oligonucleotide complementary to the mRNA transcripts to form an mRNA:DNA hybrid between the oligonucleotide and the second to fifth nucleotides of the sequence of nucleotides of the mRNA transcripts: (c) contacting the sample obtained in step (b) with nuclease (e.g., RNase H) to release the first five 5′ nucleotides of the mRNA; and (d) analyzing the sample obtained in step (c) to determine the first portion of mRNA transcripts comprising the Cap 1 structure in the mRNA sample, wherein steps (a) to (c) proceed subsequent to one another in the same reaction vessel.

In some embodiments, the invention provides a method of quantifying mRNA capping efficiency in an mRNA sample from an IVT reaction mixture, comprising steps of: (a) providing the mRNA sample, wherein the mRNA sample contains a plurality of mRNA transcripts comprising a sequence of nucleotides, wherein a first portion of the mRNA transcripts comprises a Cap 1 structure at the 5′ end of the first nucleotide of the sequence of nucleotides: (b) contacting the mRNA sample with an oligonucleotide complementary to the mRNA transcripts to form an mRNA:DNA hybrid between the oligonucleotide and the third to sixth nucleotides of the sequence of nucleotides of the mRNA transcripts: (c) contacting the sample obtained in step (b) with nuclease (e.g., RNase H) to release the first six 5′ nucleotides of the mRNA; and (d) analyzing the sample obtained in step (c) to determine the first portion of mRNA transcripts comprising the Cap 1 structure in the mRNA sample, wherein steps (a) to (c) proceed subsequent to one another in the same reaction vessel.

In some embodiments, the invention provides a method of quantifying mRNA capping efficiency in an mRNA sample from an IVT reaction mixture, comprising steps of: (a) providing the mRNA sample, wherein the mRNA sample contains a plurality of mRNA transcripts comprising a sequence of nucleotides, wherein a first portion of the mRNA transcripts comprises a Cap 1 structure at the 5′ end of the first nucleotide of the sequence of nucleotides: (b) contacting the mRNA sample with an oligonucleotide complementary to the mRNA transcripts to form an mRNA:DNA hybrid between the oligonucleotide and the fourth to seventh nucleotides of the sequence of nucleotides of the mRNA transcripts: (c) contacting the sample obtained in step (b) with nuclease (e.g., RNase H) to release the first seven 5′ nucleotides of the mRNA; and (d) analyzing the sample obtained in step (c) to determine the first portion of mRNA transcripts comprising the Cap 1 structure in the mRNA sample, wherein steps (a) to (c) proceed subsequent to one another in the same reaction vessel.

One benefit of the methods according to the present invention is that low amounts of the mRNA sample from an IVT reaction mixture are required. This is partly because no additional steps are required between steps (a) to (d). For example, the method of the invention does not require a purification step to be performed after any one of steps (a)-(c), and step (d) proceeds directly after step (c) without an intervening step. Similarly, the method of the invention does not require a further step of digestion to be performed after any one of steps (a)-(c), and step (d) proceeds directly after step (c) without an intervening step. Likewise, the method of the invention does not require a step of enrichment of the released first five, six, or seven 5′ nucleotides of the mRNA transcript, as applicable, to be performed after step (c), and step (d) proceeds directly after step (c) without an intervening step.

Amount of mRNA Input

As there are no additional steps of purification, digestion, or enrichment, the methods disclosed herein have high sample efficiency. Accordingly, in some embodiments, the method requires an input of not more than 30 to 110 pmol of IVT mRNA in the mRNA sample provided, for example 30 to 100 pmol, 30 to 90 pmol, 30 to 80 pmol, 30 to 70 pmol, 30 to 60 pmol or 30 to 50 pmol of IVT mRNA in the mRNA sample provided. For instance, in some embodiments the method requires an input of not more than 100 pmol of IVT mRNA in the mRNA sample provided. In Example 1, it is demonstrated that the method requires an input of not more than 55 pmol of IVT mRNA in the mRNA sample provided. Among other benefits, the low amounts of input mRNA required by the methods disclosed increases the amount of remaining mRNA in the IVT reaction mixture for downstream uses, such as use in formulation of a therapeutic composition.

### Assay Volume

A further benefit of the methods according to the present invention is that the method requires a small total assay volume. Accordingly, in some embodiments, the method requires a total assay volume of not more than 50 to 120 μl, for example 50 to 110 μl, 50 to 100 μl, 50 to 90 μl, 50 to 80 μl, 50 to 70 μl or 50 to 60 μl. For example, in some embodiments the method requires a total assay volume of not more than 100 μl. In Example 1, it has been found that a volume of 52 μl works well. There are many advantages to using a smaller reaction volume, including cost savings, easier scale-up and ease of transferability of the process to an automated system.

### Oligonucleotide

An oligonucleotide for use in the method of the present invention forms an mRNA:DNA hybrid between the oligonucleotide and (i) the second to fifth nucleotides, (ii) the third to sixth nucleotides, or (iii) the fourth to seventh nucleotides of the sequence of nucleotides of the mRNA transcripts. In a specific embodiment, an oligonucleotide for use in the method of the present invention may form an mRNA:DNA hybrid between the oligonucleotide and the second to fifth nucleotides of the sequence of nucleotides of the mRNA transcripts, as illustrated schematically in FIG. 3. The oligonucleotide therefore comprises at least four DNA base pairs.

The oligonucleotide for use in the method of the invention is designed not to form mRNA:DNA hybrids with off-target sites. Specific annealing of the oligonucleotide ensures that nuclease (e.g., RNase H) cleavage is directed to one site, i.e., to release the first five 5′ nucleotides, the first six 5′ nucleotides, or the first seven 5′ oligonucleotides, as appropriate. For example, releasing only the first five 5′ nucleotides with one RNase H digestion step provides a sample of low-molecular weight mRNA sequences appropriate for high-resolution analysis, in particular for determining the methylation status of the cap nucleotide.

Because the methods disclosed herein require one cleavage event, which takes place at the 5′ end of the mRNA transcript, the rest of the mRNA transcript remains intact and can be used to gather additional information regarding other aspects of the mRNA transcript, such as its length, including the length of the 3′ tail. Accordingly, in some embodiments, the mRNA sample used for the capping efficiency method as disclosed may be used in another method to analyze other aspects of the mRNA transcripts, e.g., length of the transcript or length of the 3′ tail.

Non-specific annealing of the oligonucleotide would result in a more complex mixture of released mRNA transcript nucleotides and consequent loss of resolution at the analysis stage. Therefore extra steps would be required to minimize this loss of resolution, such as the use of a purification step to remove undesired released mRNA transcript nucleotides or by simplifying the mRNA transcript to reduce off-target hybridization of the oligonucleotide, for example, through the use of an initial digestion step to shorten the mRNA transcript. In contrast, the present invention achieves high resolution without requiring an initial simplifying digestion step, purification steps, and/or transfer to a new reaction vessel.

In some embodiments, an oligonucleotide for use with the invention has the structure 5′-[R]ₙ[D]₄[R]₁-3′, wherein each R is an RNA nucleotide, each D is a DNA nucleotide, and the subscript numbers represent the number of each nucleotide. n may be adjusted to provide an oligonucleotide with a suitable melting temperature (Tₘ) and GC content (GC %). For example, a Tₘ of between about 50° C. and about 60° C., more typically between 52° C. and 58° C., may be particularly desirable for the mRNA:DNA hybrid that is formed between the oligonucleotide and the mRNA transcript. A GC % of about 40% to about 60% is typically appropriate. n can be between 10 and 20. More typically, n is between 11 and 15.

The inventors have found that an oligonucleotide with the structure: 5′-[R]₁₁[D]₄[R]₁-3′, wherein R is an RNA nucleotide, D is a DNA nucleotide, and the subscript number represents the number of each, is particularly useful.

Oligonucleotides with methylated RNA nucleotides can form more stable duplexes with the target mRNA. Moreover, oligonucleotides with methylated RNA nucleotides can readily be distinguished from mRNA fragments in a sample during subsequent analysis steps. Accordingly, in a typical embodiment, each of the RNA nucleotides of an oligonucleotide for use in the method of the invention is methylated, e.g., each RNA nucleotide may comprise a 2′-O-methyl ribose.

A skilled person in the technical field of the invention will appreciate that different 5′ UTR sequences will require the use of different oligonucleotide sequences, and it is therefore envisaged that the method of the invention can be used with any oligonucleotide that has been specifically adapted for binding to the a 5′ UTR or an mRNA transcript such that an mRNA:DNA hybrid between the oligonucleotide and the second to fifth nucleotides, the third to sixth nucleotides, or the fourth to seventh nucleotides, respectively, of the sequence of nucleotides of the mRNA transcript (i.e., not counting the cap nucleotide) is formed.

For example, in some embodiments, an exemplary 5′ UTR comprises the sequence: GGACAGAUCGCCUGGAGACGCCAUCCACGCUGUUUUGACCUCCAUAGAAGACA CCGGGACCGAUCCAGCCUCCGCGGCCGGGAACGGUGCAUUGGAACGCGGAUUC CCCGUGCCAAGAGUGACUCACCGUCCUUGACACG [SEQ ID NO: 2]. In some embodiments, the 5′ UTR comprises the sequence: AGACAGAUCGCCUGGAGACGCCAUCCACGCUGUUUUGACCUCCAUAGAAGACA CCGGGACCGAUCCAGCCUCCGCGGCCGGGAACGGUGCAUUGGAACGCGGAUUC CCCGUGCCAAGAGUGACUCACCGUCCUUGACACG [SEQ ID NO: 3]. In some embodiments, the 5′ UTR comprises the sequence: GAGAAUAAACUAGUAUUCUUCUGGUCCCCACAGACUCAGAGAGAACCCGCCAC C [SEQ ID NO: 4]. In some embodiments, the 5′ UTR comprises the sequence: GGGAAAUAAGAGAGAAAAGAAGAGUAAGAAGAAAUAUAAGAGCCACC [SEQ ID NO: 5]. In some embodiments, the 5′ UTR comprises the sequence: GGAGAAAGCUUACC [SEQ ID NO: 6]. In some embodiments, the 5′ UTR comprises the sequence: GGGAAAUAAGAGAGAAAAGAAGAGUAAGAAGAAAUAUAAGACCCCGGCGCCG CCACC [SEQ ID NO: 7]. In some embodiments, the 5′ UTR comprises the sequence: AGGGUCAAGAUUAGAGAACGGUCGUAGCAUUAUCGGAGGUUCUGCCACC [SEQ ID NO: 8]. In some embodiments, the 5′ UTR comprises the sequence: GGGUCAAGAUUAGAGAACGGUCGUAGCAUUAUCGGAGGUUCUGCCACC [SEQ ID NO: 9].

In some embodiments, the 5′ UTR comprises a sequence selected from SEQ ID NOs: 1-1363, SEQ ID NO: 1395, SEQ ID NO: 1421 and SEQ ID NO: 1422 of WO2013/143700. In some embodiments, the 5′ UTR comprises a sequence selected from SEQ ID NOs: 25 to 30 and SEQ ID NOs: 319 to 382 of WO2016/107877. In some embodiments, the 5′ UTR comprises a sequence selected from SEQ ID NOs: 1 to 151 of WO2017/036580. In some embodiments, the 5′ UTR comprises a sequence selected from SEQ ID NOs: 4276-4282 of WO2014/164253.

### Exemplary Oligonucleotide

Exemplary oligonucleotides suitable for use in the methods of the invention include the following: 5′-UCCAGGCGAUCTGTCC-3′ (SEQ ID NO: 10), 5′-UUCUCUCUUAUTTCCC-3′ (SEQ ID NO: 11), 5′-CUUUUCUCUCUUAUTTCCC-3″ (SEQ ID NO: 12), 5′-CAGAAGAAUACTAGTU-3′ (SEQ ID NO: 13), 5′-GGACCAGAAGAAUACTAGTU-3′ (SEQ ID NO: 14), 5′-CGUUCUCUAAUCUUGACCCU-3′ (SEQ ID NO: 15), 5′-CUCUAAUCUUGACCCU-3′ (SEQ ID NO: 16), 5′-CCGUUCUCUAAUCUUGACCC-3′ (SEQ ID NO: 17), and 5′-CUCUAAUCUUGACCC-3′ (SEQ ID NO: 18). The underlined nucleotides are DNA. The remaining nucleotides are RNA. In a typical embodiment, the RNA nucleotides are methylated, e.g., they may comprise a 2′-O-methyl ribose.

An exemplary oligonucleotide that was found to be particularly suitable for use with the invention has the sequence: 5′-mUmCmCmAmGmGmCmGmAmUmCTGTCmC-3′ [SEQ ID NO: 1]. This oligonucleotide can be used with mRNAs comprising the following 5′ untranslated region (5′ UTR): GGACAGAUCGCCUGGAGACGCCAUCCACGCUGUUUUGACCUCCAUAGAAGAC ACCGGGACCGAUCCAGCCUCCGCGGCCGGGAACGGUGCAUUGGAACGCGGAUU CCCCGUGCCAAGAGUGACUCACCGUCCUUGACACG [SEQ ID NO: 2]. The oligonucleotide binding site in the 5′ UTR is shown in bold.

Another exemplary oligonucleotide for use with the invention has the sequence: 5′-mUmUmCmUmCmUmCmUmUmAmUTTCCmC-3′ [SEQ ID NO: 19]. This oligonucleotide can be used with mRNAs comprising the following 5′ untranslated regions (5′ UTRs): GGGAAAUAAGAGAGAAAAGAAGAGUAAGAAGAAAUAUAAGACCCCGGCGCCG CCACC [SEQ ID NO: 7] and GGGAAAUAAGAGAGAAAAGAAGAGUAAGAAGAAAUAUAAGAGCCACC [SEQ ID NO: 5]. The oligonucleotide binding sites in the 5′ UTRs are shown in bold.

Another exemplary oligonucleotide for use with the invention has the sequence: 5′-mCmUmUmUmUmCmUmCmUmCmUmUmAmUTTCCmC-3′ [SEQ ID NO: 20]. This oligonucleotide can be used with mRNAs comprising the following 5′ untranslated regions (5′ UTRs): GGGAAAUAAGAGAGAAAAGAAGAGUAAGAAGAAAUAUAAGACCCCGGCGCCG CCACC [SEQ ID NO: 7] and GGGAAAUAAGAGAGAAAAGAAGAGUAAGAAGAAAUAUAAGAGCCACC [SEQ ID NO: 5]. The oligonucleotide binding sites in the 5′ UTRs are shown in bold.

Another exemplary oligonucleotide for use with the invention has the sequence: 5′-mCmAmGmAmAmGmAmAmUmAmCTAGTmU-3′ [SEQ ID NO: 21]. This oligonucleotide can be used with mRNAs comprising the following 5′ untranslated region (5′ UTR): AACUAGUAUUCUUCUGGUCCCCACAGACUCAGAGAGAACCCGCCACC [SEQ ID NO: 22]. The oligonucleotide binding site in the 5′ UTR is shown in bold.

Another exemplary oligonucleotide for use with the invention has the sequence: 5′-mGmGmAmCmCmAmGmAmAmGmAmAmUmAmCTAGTmU-3′ [SEQ ID NO: 23]. This oligonucleotide can be used with mRNAs comprising the following 5′ untranslated region (5′ UTR): AACUAGUAUUCUUCUGGUCCCCACAGACUCAGAGAGAACCCGCCACC [SEQ ID NO: 22]. The oligonucleotide binding site in the 5′ UTR is shown in bold.

Another exemplary oligonucleotide for use with the invention has the sequence: 5′-mCmGmUmUmCmUmCmUmAmAmUmCmUmUmGACCCmU-3′ [SEQ ID NO: 24]. This oligonucleotide can be used with mRNAs comprising the following 5′ untranslated region (5′ UTR): AGGGUCAAGAUUAGAGAACGGUCGUAGCAUUAUCGGAGGUUCUGCCACC [SEQ ID NO: 8]. The oligonucleotide binding site in the 5′ UTR is shown in bold.

Another exemplary oligonucleotide for use with the invention has the sequence: 5′-mCmUmCmUmAmAmUmCmUmUmGACCCmU-3′ [SEQ ID NO: 25]. This oligonucleotide can be used with mRNAs comprising the following 5′ untranslated region (5′ UTR): AGGGUCAAGAUUAGAGAACGGUCGUAGCAUUAUCGGAGGUUCUGCCACC [SEQ ID NO: 8]. The oligonucleotide binding site in the 5′ UTR is shown in bold.

Another exemplary oligonucleotide for use with the invention has the sequence: 5′-mCmCmGmUmUmCmUmCmUmAmAmUmCmUmUGACCmC-3′ [SEQ ID NO: 26]. This oligonucleotide can be used with mRNAs comprising the following 5′ untranslated region (5′ UTR): GGGUCAAGAUUAGAGAACGGUCGUAGCAUUAUCGGAGGUUCUGCCACC [SEQ ID NO: 9]. The oligonucleotide binding site in the 5′ UTR is shown in bold.

Another exemplary oligonucleotide for use with the invention has the sequence: 5′-mCmUmCmUmAmAmUmCmUmUGACCmC-3′ [SEQ ID NO: 27]. This oligonucleotide can be used with mRNAs comprising the following 5′ untranslated region (5′ UTR): GGGUCAAGAUUAGAGAACGGUCGUAGCAUUAUCGGAGGUUCUGCCACC [SEQ ID NO: 9]. The oligonucleotide binding site in the 5′ UTR is shown in bold.

In the oligonucleotide sequences, mU represents uridine with a 2′-O-methyl ribose; mC represents cytidine with a 2′-O-methyl ribose; mA represents adenine with a 2′-O-methyl ribose; mG represents guanidine with a 2′-O-methyl ribose; A represents deoxyadenine; T represents deoxythymidine; G represents deoxyguanidine and C represents deoxycytidine.

### Nuclease

In accordance with the present invention, a nuclease that can catalyze the cleavage of the RNA and/or DNA strand in a RNA:DNA hybrid substrate may be used for cleavage of the first nucleotides from the untranslated region of mRNA molecules in the mRNA sample; such nuclease is non-sequence specific. In some embodiments, more than one nucleases are used in a single capping efficiency quantification.

In some embodiments, a suitable nuclease is RNase H or any enzyme with RNase H like enzymatic activity. In some embodiments, a suitable nuclease is RNase S1 or any enzyme with RNase S1 like enzymatic activity. In other embodiments, a suitable nuclease is selected from Benzonase®, nuclease P1, phosphodiester, RNase A and RNase T1. In some embodiments, multiple nucleases are used; for example RNase H and an S1 nuclease.

In one embodiment, the nuclease is RNase H.

### RNase H

RNases H form a ubiquitous enzyme family that is divided into two distinct phylogenetic subtypes, Type 1 and Type 2, either of which may be used in particular embodiments of the invention. The RNases H are unified by the common ability to bind a single-stranded (ss) RNA that is hybridized to a complementary DNA single strand, and then degrade the RNA portion of the RNA:DNA hybrid. While the RNases H have been implicated in DNA replication and recombination, and repair, their physiological roles are not completely understood. In vitro, the enzymes will also bind double-stranded (ds) DNA, SSDNA, ssRNA, and dsRNA, albeit with lower affinities than they bind to RNA:DNA hybrids. Due to the ubiquity of the enzyme, there are several sequences for RNase H known in the literature, each of which vary somewhat in their amino acid sequences. U.S. Pat. No. 5,268,289 discloses a thermostable RNase H, as does U.S. Pat. No. 5,500,370. U.S. Pat. No. 6,376,661 discloses a human RNase H and compositions and uses thereof. U.S. Pat. No. 6,001,652 discloses a human type 2 RNase H. U.S. Pat. No. 6,071,734 discloses RNase H from HBV polymerase. All of these RNases H may be used in one more embodiments of the invention.

### Assay Run Time

Another benefit of the methods according to the present invention is that the reaction steps take a short amount of time to complete. Accordingly, in some embodiments, completion of the steps of contacting the mRNA sample with an oligonucleotide and contacting the sample with nuclease (e.g. RNase H) to release the first five, six, or seven nucleotides of the sequence of nucleotides of the mRNA transcripts takes less than 90 minutes. In some embodiments, the steps are performed in 40-100 minutes, 45-90 minutes, 50-80 minutes, 55-70 minutes or 60-70 minutes.

As illustrated in Example 1, the steps of contacting the mRNA sample with an oligonucleotide and contacting the sample with nuclease (e.g., RNase H) to release the first five, six, or seven nucleotides of the sequence of nucleotides of the mRNA transcripts are performed in 60 minutes. Accordingly, in particular embodiments of the invention, the steps of contacting the mRNA sample with an oligonucleotide and then contacting that sample with nuclease (e.g., RNase H) requires not more than 60 minutes, or less than 65 minutes, to complete.

### Automation

One benefit of the methods according to the present invention is that the steps of contacting the mRNA sample with an oligonucleotide complementary to the mRNA transcripts, contacting the sample with nuclease (e.g., RNase H) and analyzing the sample to determine the portion of mRNA transcripts comprising the Cap 1 structure can be performed on an automated system. Automation can further improve the time efficiency of the methods disclosed herein. Automation also has benefits for scale-up of the methods for determining capping efficiency disclosed herein. In some embodiments, automation comprises use of an automated liquid handling system. In a specific embodiment, the automated liquid handling systems processes about 100 samples at a time. For example, in a particular embodiment, the automated liquid handling system is adapted for use with a multi-well plate (e.g., a 96-well plate). Commercially available liquid handling systems include Microlab VANTAGE 1.3, Microlab STAR, Microlab NIMBUS96 or Microlab Prep (all Hamilton). Similar systems are also available from Tecan (e.g., the Tecan Fluent or Freedom EVO liquid handling platforms). Other automated liquid handlers will be known to the skilled person.

### Chromatographic Separation

The nuclease treated (e.g., RNase H-treated) sample is analyzed to determine the portion of mRNA transcripts comprising the Cap 1 structure in the mRNA sample. In a typical embodiment, the invention utilizes chromatography to resolve nucleotides corresponding to the first five, six, or seven nucleotides of the sequence of nucleotides of the mRNA transcript comprising Cap 1 from nucleotides corresponding to the first five, six, or seven nucleotides of the sequence of nucleotides of the mRNA transcript comprising Cap 0 and/or Cap G and/or nucleotides corresponding to the first five, six, or seven nucleotides of the sequence of nucleotides of the mRNA transcript lacking a cap structure.

In some embodiments, mRNA samples comprising the first five, six, or seven nucleotides of the sequence of nucleotides of the mRNA transcript can be subjected to thin-layer chromatography (“TLC”).

In particular embodiments, the analysis according to step (d) comprises analyzing the sample obtained in step (c) by High Performance Liquid Chromatography (HPLC). The term “HPLC” as used herein also relates to Ultra-High Performance Liquid Chromatography (UHPLC).

In a specific embodiment, a method in accordance with the invention uses UHPLC for the analysis performed in step (d). UHPLC typically uses columns packed with particles smaller than 2 μm (e.g., 1.7 μm). UHPLC systems usually employ a pressure higher than 6000 psi (e.g., up to 15,000 psi), resulting in high flow rates for increased speed. In combination with the small particle size, UHPLC provides superior resolution and sensitivity relative to conventional HPLC systems (see, e.g., Swartz, M. E., “Ultra Performance Liquid Chromatography (UPLC): an introduction”, SEPARATION SCIENCE REDEFINED (2005)).

HPLC may comprise different methods of sample separation, depending on the column used. For example, in some embodiments, a suitable chromatographic column is selected from the group consisting of an anion-exchange column, a cation-exchange column, a reverse phase HPLC column, a hydrophobic interaction column, a size exclusion column or combinations thereof. In particular embodiments, a reverse phase HPLC column is used, e.g. a reverse phase UHPLC column. In a specific embodiment, the analysis according to step (d) comprises analyzing the sample obtained in step (c) with a C18 reverse-phase UHPLC column. The inventors have found a C18 2.1×100 mm column to be particularly useful. In some embodiments, it may be desirable to heat the sample (e.g., to about 50° C.) or apply the sample to a heated chromatographic column.

As will be known by those skilled in the art, ion exchangers (e.g., anion exchangers and/or cation exchangers) may be based on various materials with respect to the matrix as well as to the attached charged groups. For example, the following matrices may be used, in which the materials mentioned may be more or less crosslinked: an agarose-based matrix (such as Sepharose™ CL-6B, Sepharose™ Fast Flow and Sepharose™ High Performance), cellulose-based matrix (such as DEAE Sephacel®), dextran-based matrix (such as SEPHADEX®), silica-based matrix and synthetic polymer-based matrix.

An ion exchange resin can be prepared according to known methods. Typically, an equilibration buffer, which allows the resin to bind its counter ions, can be passed through the ion exchange resin prior to loading the sample onto the resin. Conveniently, the equilibration buffer can be the same as the loading buffer, but this is not required. In one embodiment, the ion exchange resin can be regenerated with a regeneration buffer after elution of the sample, such that the column can be re-used. Generally, the salt concentration and/or pH of the regeneration buffer can be such that substantially all contaminants and all remaining sample are eluted from the ion exchange resin. Generally, the regeneration buffer has a very high salt concentration for eluting contaminants and nucleotides from the ion exchange resin.

In some embodiments, the sample obtained in step (c) is subjected to anion exchange chromatography for the analysis according to step (d). High-resolution analysis of nucleotides may be performed as described in Ausser, W. A., et al., “High-resolution analysis and purification of synthetic oligonucleotides with strong anion-exchange HPLC”, BIOTECHNIQUES, 19: 136-139 (1995). For the anion exchange resin, the charged groups which are covalently attached to the matrix can be, for example, diethylaminoethyl (DEAE), quaternary aminoethyl (QAE), and/or quaternary ammonium (Q). In some embodiments, the anion exchange resin employed is a Q Sepharose column. The anion exchange chromatography can be performed, for example, using, e.g., Q Sepharose™ Fast Flow, Q Sepharose™ High Performance, Q Sepharose™ XL, Capto™ Q, DEAE, TOYOPEARL Gigacap® Q, Fractogel R TMAE (trimethylaminoethyl, a quarternary ammonia resin), Eshmuno™ Q, Nuvia™ Q, or UNOsphere™ Q. Other anion exchangers include quaternary amine resins or “Q-resins” (e.g., Capto™-Q, Q-Sepharose®, QAE Sephadex®): diethylaminoethane resins (e.g., DEAE-Trisacryl®, DEAE Sepharose®, benzoylated naphthoylated DEAE, diethylaminoethyl Sephacel®); Amberjet® resins; Amberlyst® resins: Amberlite® resins (e.g., Amberlite® IRA-67, Amberlite® strongly basic, Amberlite® weakly basic), cholestyramine resin, ProPac® resins (e.g., ProPac® SAX-10, ProPac® WAX-10, ProPac® WCX-10); TSK-GEL® resins (e.g., TSKgel DEAE-NPR; TSKgel DEAE-5PW); and Acclaim® resins.

Typical mobile phases for anionic exchange chromatography include polar solutions, such as water, acetonitrile, organic alcohols such as methanol, ethanol, and isopropanol, or solutions containing 2-(N-morpholino)-ethanesulfonic acid (MES). Thus, in certain embodiments, the mobile phase includes about 0%, 1%, 2%, 4%, 6%, 8%, 10%, 12%, 14%, 16%, 18%, 20%, 25%, 30%, 35%, 40%, 45%, 50%, 55%, 60%, 65%, 70%, 75%, 80%, 85%, 90%, 95%, or about 100% polar solution. In certain embodiments, the mobile phase comprises between about 1% to about 100%, about 5% to about 95%, about 10% to about 90%, about 20% to about 80%, about 30% to about 70%, or about 40% to about 60% polar solution at any given time during the course of the separation.

In general, uncapped mRNA adsorbs onto the fixed positive charge of a strong anion exchange column and a gradient of increasing ionic strength using a mobile phase at a predetermined flow rate elutes capped species (the cap bearing a positive charge) from the column in proportion to the strength of their ionic interaction with the positively charged column. More negatively charged (more acidic) mRNA nucleotides lacking a cap structure elute later than less negatively charged (less acidic) capped species.

In some embodiments, the analysis according to step (d) comprises subjecting the same obtained in step (c) to cation exchange chromatography, e.g., sulfopropyl (SP) cation exchange chromatography. Other cation chromatography membranes or resins can be used, for example, a MUSTANG™ S membrane, an S-Sepharose™ resin, or a Blue Sepharose™ resin.

In some embodiments, the analysis according to step (d) comprises subjecting the same obtained in step (c) to hydrophobic interaction chromatography (HIC). Hydrophobic interaction chromatography utilizes the attraction of a given molecule for a polar or non-polar environment, and in terms of nucleic acids, this propensity is governed by the hydrophobicity or hydrophilicity of nucleotides and modifications thereon. Thus, nucleic acids are fractionated based upon their varying degrees of attraction to a hydrophobic matrix, typically an inert support with alkyl linker arms of 2-18 carbons in chain length. The stationary phase comprises small non-polar groups (butyl, octyl, or phenyl) attached to a hydrophilic polymer backbone (e.g., cross-linked Sepharose™, dextran, or agarose). In some embodiments, the hydrophobic interaction chromatography includes phenyl chromatography. In other embodiments, the hydrophobic interaction chromatography includes butyl chromatography or octyl chromatography.

In some embodiments, the analysis according to step (d) comprises subjecting the same obtained in step (c) to reverse phase-HPLC. Reversed phase HPLC comprises a non-polar stationary phase and a moderately polar mobile phase. In some embodiments, the stationary phase is a silica which has been treated with, for example, RMe₂SiCl, where R is a straight chain alkyl group such as C₁₈H₃₇ or C₈H₁₇. The retention time is therefore longer for molecules which are more non-polar in nature, allowing polar molecules to elute more readily. Retention time is increased by the addition of polar solvent to the mobile phase and decreased by the addition of more hydrophobic solvent. Other parameters that can also be altered include mobile phase flow rate and temperature. The characteristics of the specific RNA molecule as an analyte may play an important role in its retention characteristics. In general, an analyte having more non-polar functional groups (e.g., methyl groups) results in a longer retention time because it increases the molecule's hydrophobicity. Protocols for high resolution of RNA species using reverse phase-HPLC, which may be adapted for use in embodiments of the invention, are known in the art (see, e.g., U.S. Pat. No. 8,383,340; Gilar, M., “Analysis and purification of synthetic oligonucleotides by reversed-phase high-performance liquid chromatography with photodiode array and mass spectrometry detection”, ANAL. BIOCHEM., 298: 196-206 (2001)). In some embodiments, a triethylammonium acetate (TEAA) buffer is used with UV detection for separation and characterization of the first five, six, or seven nucleotides of the sequence of nucleotides of the mRNA transcript comprising a cap.

In some embodiments, the analysis according to step (d) of the method utilizes a combination of one or more chromatographic separation methods disclosed herein. For example, particular embodiments of the invention may utilize reverse-phase ion-pair chromatography, whereby separations are based on both hydrophobicity and on the number of anions associated with the molecule. Matrices can be silica-based (e.g., Murray et al., ANAL. BIOCHEM., 218:177-184 (1994)). Non-porous, inert polymer resins may be used in particular embodiments (see, e.g., Huber, C. G., “High-resolution liquid chromatography of oligonucleotides on nonporous alkylated styrene-divinylbenzene copolymers”, ANAL. BIOCHEM., 212: 351-358 (1993)).

In certain embodiments, the analysis according to step (d) may comprise chromatography (e.g., HPLC, in particular UHPLC) combined with mass spectrometry (“MS”). Suitable LC-MS methods are known in the art. Such methods typically use aqueous triethylamine-hexafluoroisopropanol (TEA HFIP) buffers compatible with MS detection (Apffel, A., et al., “New procedure for the use of HPLC-ESI MS for the analysis of nucleotides and oligonucleotides”, J. Chromatogr. A, 777: 3-21 (1997)). For example, an optimized TEA-HFIP mobile phase may be used for LC-MS separation and characterization of the first five, six, or seven nucleotides of the sequence of nucleotides of the mRNA transcript comprising a cap. In particular embodiments, the aqueous buffer comprises 100 mM HFIP and 8. 6 mM TEA at pH 8.3. In specific embodiments, a mobile phase comprising an aqueous solution of 100 mM HFIP/8.6 mM TEA, pH 8.3, combined with up to 50% methanol is used in a method in accordance with the invention. Alternatively, a triethylammonium bicarbonate mobile phase may be used for oligonucleotide separation with post-column acetonitrile addition to the eluent. The ion-pairing buffer may be chosen to give the best MS detection sensitivity. In certain embodiments, the analysis according to step (d) may comprise chromatography (e.g., HPLC) combined with tandem mass spectrometry (LC-MS/MS). The inventors have found ultra-high performance liquid chromatography-quadrupole time-of-flight mass spectrometry (UHPLC/Q-TOF-MS) to be a particularly suitable method of analysis according to step (d) of the invention. Accordingly, in a particular embodiment, a method in accordance with the invention uses ultra-high performance liquid chromatography-quadrupole time-of-flight mass spectrometry (UHPLC/Q-TOF-MS) to perform the analysis in step (d).

In some embodiments, analysis of the sample according to step (d) by MS or LC-MS comprises analysis over a scan range of 200-6000 m/z, or 250-5000 m/z, or 300-4000 m/z. In particular embodiments, analysis of the sample according to step (d) by MS or LC-MS comprises analysis over a scan range of 400-3200 m/z.

### Methylation Profile

In certain embodiments, the mRNA transcripts comprising cap structures are characterized by a methylation profile. In some embodiments, a “methylation profile” refers to a set of values corresponding to the amount of the first five, six, or seven nucleotides of the sequence of nucleotides of the mRNA transcript that elute from a chromatography column at a point in time after addition to the column of a mobile phase. As described above, the retention time for methylated caps and penultimate nucleotides, which are more non-polar in nature, is increased relative to polar molecules, which elute more readily. Accordingly, in some embodiments, retention time of the first five, six, or seven nucleotides released in step (c) of the method of the invention may be increased by the addition of a polar solvent to the mobile phase used during a chromatographic analysis step performed in step (d). In some embodiments, retention time of the first five, six, or seven nucleotides released in step (c) of the method of the invention may be decreased by the addition of a hydrophobic solvent to the mobile phase used during a chromatographic analysis step performed in step (d).

### Peak Analysis

In some embodiments, the analysis according to step (d) of the methods disclosed herein comprises automated integration of respective peak areas in a chromatogram. For example, data may be presented as area percent value, which refers to the percentage of a particular species' integrated peak area relative to the total integrated peak area of the entire chromatogram.

In some embodiments, the analysis according to step (d) of the methods disclosed herein comprises comparison of the respective peaks in the chromatogram(s) following HPLC analysis. In particular embodiments, the analysis according to step (d) of the methods disclosed herein comprises comparison of the total ion counts of the respective peaks in the chromatogram(s) following mass spectrometry analysis (e.g., LC-MS).

In some embodiments, the portion of the first five, six, or seven nucleotides of the sequence of nucleotides of the mRNA transcripts comprising a Cap 1 structure is determined relative to the portion of the first five, six, or seven nucleotides of the sequence of nucleotides of the mRNA transcripts comprising other cap structures (e.g., Cap 0 or Cap G), or lacking a cap structure.

In a particular embodiment, the portion of the first five nucleotides of the sequence of nucleotides of the mRNA transcripts comprising a Cap 1 structure is determined relative to the portion of the first five nucleotides of the sequence of nucleotides of the mRNA transcripts comprising other cap structures (e.g., Cap 0 or Cap G), or lacking a cap structure.

### Control Sample

In some embodiments, the analysis according to step (d) of the method of the invention is performed with reference to a one or more control samples. For example, in some embodiments, the analysis according to step (d) of the method of the invention is performed with reference to an uncapped control sample comprising a synthesized oligonucleotide corresponding to the first five, six, or seven nucleotides, respectively, of the sequence of nucleotides of the mRNA in the mRNA sample according to the invention.

In other embodiments, a suitable control sample may comprise (i) a sequence of five, six, or seven nucleotides, as appropriate, comprising a 5′ cap structure, and (ii) a sequence of five, six, or seven nucleotides, as appropriate, lacking a cap structure, at a defined ratio (e.g., 9:1) to provide a reference. For example, where the oligonucleotide is designed for the enzymatic release of the first five 5′ nucleotides of an mRNA transcript, a control sample may contain (i) a sequence of five nucleotides comprising a 5′ Cap 1 structure, and (ii) a sequence of five nucleotides lacking a cap structure, at a defined ratio to provide a reference. In a specific embodiment, a control sample contains (i) a sequence of five nucleotides comprising a 5′ Cap 1 structure, and (ii) a sequence of five nucleotides lacking a cap structure at a ratio of 9:1.

In some embodiments, the one or more control samples are used in a calibration step as part of the analysis according to step (d) of the method of the invention. In some embodiments, a control sample is used to monitor machine calibration by measuring the elution time and/or peak intensity of the control sample. In some embodiments, a control sample comprising a synthesized oligonucleotide corresponding to the first five, six, or seven nucleotides, respectively, of the sequence of nucleotides of the mRNA is analyzed according to step (d) of the method of the invention to monitor machine calibration.

In some embodiments, the one or more control samples are run in parallel to the analysis of the sample obtained according to step (c) to provide a reference chromatogram. In some embodiments, the control samples are modified, for example deuterated or radiolabeled, such that the sample obtained according to step (c) can be spiked with one or more control samples for simultaneous measurement of the sample obtained according to step (c) and the one or more control samples.

Given the high resolution achieved with the methods of the invention, which, e.g., can readily distinguish between first five nucleotides of the sequence of nucleotides of the mRNA transcript including a cap and first five nucleotides of the sequence of nucleotides of the mRNA transcript not including a cap as well as between different cap structures (e.g., Cap 0, Cap G and Cap 1), the use of a control sample is not strictly necessary. Accordingly, in a particular embodiment of the invention, no control sample is used in step (d) to analyze the sample obtained in step (c) of the method of the invention.

Further Processing of the mRNA Transcripts

In some embodiments, the method of the invention is integrated into a manufacturing process and the result of step (d) is used to determine whether the IVT reaction mixture from which the mRNA sample was taken is processed further.

By “processed further”, it is meant that the reaction mixture will be moved to the next step in a given manufacturing process. For example, further processing may comprise purification, for example to remove certain components of the IVT reaction mixture. Further processing may also include formulation of the mRNA transcripts, for example, by encapsulation in a lipid nanoparticle. For example, further processing may comprise use of the IVT reaction mixture in preparation of a therapeutic composition.

In specific embodiments, further processing may comprise addition of a 3′ poly(A) tail. In some embodiments, a 3′ poly(A) tail of approximately 200 nucleotides in length (as determined by gel electrophoresis) is incorporated through the addition of ATP in conjunction with PolyA polymerase. In some embodiments, the poly(A) tail is approximately 100-250 nucleotides in length. In some embodiments, the poly(A) tail is about 50-300 nucleotides in length. In some embodiments, the mRNA transcripts in the IVT reaction mixture include 5′ and 3′ untranslated regions.

In some embodiments, further processing comprises further quality control steps. For example, the IVT reaction mixture may be used as a source for further mRNA sample that can be analyzed regarding other aspects of the mRNA transcript, e.g. length of the mRNA transcript. Further processing may comprise determining the purity of the mRNA sample.

Typically, an IVT reaction mixture that is determined to comprise an amount of mRNA transcripts comprising a Cap 1 structure above a threshold value is taken to be processed further. In some embodiments, mRNA transcripts comprising the Cap 1 structure are at least 80% of the sample for the IVT reaction mixture to be processed further. In some embodiments, mRNA transcripts comprising the Cap 1 structure are at least 85% of the sample for the IVT reaction mixture to be processed further. In some embodiments, mRNA transcripts comprising the Cap 1 structure are at least 90% of the sample for the IVT reaction mixture to be processed further. In some embodiments, mRNA transcripts comprising the Cap 1 structure are at least 91% of the sample for the IVT reaction mixture to be processed further. In some embodiments, mRNA transcripts comprising the Cap 1 structure are at least 92% of the sample for the IVT reaction mixture to be processed further. In some embodiments, mRNA transcripts comprising the Cap 1 structure are at least 93% of the sample for the IVT reaction mixture to be processed further. In some embodiments, mRNA transcripts comprising the Cap 1 structure are at least 94% of the sample for the IVT reaction mixture to be processed further. In a particular embodiment, mRNA transcripts comprising the Cap 1 structure are at least 95% of the sample for the IVT reaction mixture to be processed further. In some embodiments, mRNA transcripts comprising the Cap 1 structure are at least 96% of the sample for the IVT reaction mixture to be processed further. In some embodiments, mRNA transcripts comprising the Cap 1 structure are at least 97% of the sample for the IVT reaction mixture to be processed further. In some embodiments, mRNA transcripts comprising the Cap 1 structure are at least 98% of the sample for the IVT reaction mixture to be processed further. In some embodiments, mRNA transcripts comprising the Cap 1 structure are at least 99% of the sample for the IVT reaction mixture to be processed further.

Accordingly, an IVT reaction mixture that is determined to comprise an amount of mRNA transcripts comprising a Cap structure other than Cap 1 is taken to be processed further only if the Cap structure other than Cap 1 is present below a threshold value. In some embodiments, mRNA transcripts comprising the Cap 0 structure are not more than 20% of the sample for the IVT reaction mixture to be processed further. In some embodiments, mRNA transcripts comprising the Cap 0 structure are not more than 15% of the sample for the IVT reaction mixture to be processed further. In some embodiments, mRNA transcripts comprising the Cap 0 structure are not more than 10% of the sample for the IVT reaction mixture to be processed further. In some embodiments, mRNA transcripts comprising the Cap 0 structure are not more than 9% of the sample for the IVT reaction mixture to be processed further. In some embodiments, mRNA transcripts comprising the Cap 0 structure are not more than 8% of the sample for the IVT reaction mixture to be processed further. In some embodiments, mRNA transcripts comprising the Cap 0 structure are not more than 7% of the sample for the IVT reaction mixture to be processed further. In some embodiments, mRNA transcripts comprising the Cap 0 structure are not more than 6% of the sample for the IVT reaction mixture to be processed further. In some embodiments, mRNA transcripts comprising the Cap 0 structure are not more than 5% of the sample for the IVT reaction mixture to be processed further. In some embodiments, mRNA transcripts comprising the Cap 0 structure are not more than 4% of the sample for the IVT reaction mixture to be processed further. In some embodiments, mRNA transcripts comprising the Cap 0 structure are not more than 3% of the sample for the IVT reaction mixture to be processed further. In some embodiments, mRNA transcripts comprising the Cap 0 structure are not more than 2% of the sample for the IVT reaction mixture to be processed further. In some embodiments, mRNA transcripts comprising the Cap 0 structure are not more than 1% of the sample for the IVT reaction mixture to be processed further.

In some embodiments, mRNA transcripts comprising the Cap G structure are not more than 20% of the sample for the IVT reaction mixture to be processed further. In some embodiments, mRNA transcripts comprising the Cap G structure are not more than 15% of the sample for the IVT reaction mixture to be processed further. In some embodiments, mRNA transcripts comprising the Cap G structure are not more than 10% of the sample for the IVT reaction mixture to be processed further. In some embodiments, mRNA transcripts comprising the Cap G structure are not more than 9% of the sample for the IVT reaction mixture to be processed further. In some embodiments, mRNA transcripts comprising the Cap G structure are not more than 8% of the sample for the IVT reaction mixture to be processed further. In some embodiments, mRNA transcripts comprising the Cap G structure are not more than 7% of the sample for the IVT reaction mixture to be processed further. In some embodiments, mRNA transcripts comprising the Cap G structure are not more than 6% of the sample for the IVT reaction mixture to be processed further. In some embodiments, mRNA transcripts comprising the Cap G structure are not more than 5% of the sample for the IVT reaction mixture to be processed further. In some embodiments, mRNA transcripts comprising the Cap G structure are not more than 4% of the sample for the IVT reaction mixture to be processed further. In some embodiments, mRNA transcripts comprising the Cap G structure are not more than 3% of the sample for the IVT reaction mixture to be processed further. In some embodiments, mRNA transcripts comprising the Cap G structure are not more than 2% of the sample for the IVT reaction mixture to be processed further. In some embodiments, mRNA transcripts comprising the Cap G structure are not more than 1% of the sample for the IVT reaction mixture to be processed further.

Commonly, an IVT reaction mixture that is determined to comprise an amount of uncapped mRNA transcripts is taken to be processed further only if the amount is below a threshold value. In a specific embodiment, uncapped mRNA transcripts are not more than 20% of the sample for the ITV reaction mixture to be processed further. In a specific embodiment, uncapped mRNA transcripts are not more than 15% of the sample for the ITV reaction mixture to be processed further. In a specific embodiment, uncapped mRNA transcripts are not more than 10% of the sample for the ITV reaction mixture to be processed further. In a specific embodiment, uncapped mRNA transcripts are not more than 9% of the sample for the ITV reaction mixture to be processed further. In a specific embodiment, uncapped mRNA transcripts are not more than 8% of the sample for the ITV reaction mixture to be processed further. In a specific embodiment, uncapped mRNA transcripts are not more than 7% of the sample for the ITV reaction mixture to be processed further. In a specific embodiment, uncapped mRNA transcripts are not more than 6% of the sample for the ITV reaction mixture to be processed further. In a specific embodiment, uncapped mRNA transcripts are not more than 5% of the sample for the ITV reaction mixture to be processed further. In a specific embodiment, uncapped mRNA transcripts are not more than 4% of the sample for the ITV reaction mixture to be processed further. In a specific embodiment, uncapped mRNA transcripts are not more than 3% of the sample for the ITV reaction mixture to be processed further. In a specific embodiment, uncapped mRNA transcripts are not more than 2% of the sample for the ITV reaction mixture to be processed further. In a specific embodiment, uncapped mRNA transcripts are not more than 1% of the sample for the ITV reaction mixture to be processed further.

Although an IVT reaction mixture determined to comprise less than 90% mRNA transcripts comprising Cap 1 will not be taken to be processed further, the mRNA sample or the IVT reaction mixture may be used for other purposes, for instance to determine the cause of the insufficient generation of Cap 1.

### Kits

The present invention further provides kits comprising various reagents and materials useful for carrying out inventive methods according to the present invention. The quantitative procedures described herein may be performed by diagnostic laboratories, experimental laboratories, or commercial laboratories. The invention provides kits which can be used in these different settings.

For detecting/quantifying mRNA capping efficiency, kits may comprise a oligonucleotide as disclosed herein that specifically anneals adjacent to an mRNA cap of the target mRNA transcript to form an mRNA:DNA hybrid between the oligonucleotide and the second to fifth nucleotides, the third to sixth nucleotides, or the fourth to seventh nucleotides, respectively, of the sequence of nucleotides of the mRNA transcript. Kits may also comprise a nuclease for production of the first five, six, or seven nucleotides, respectively, of the sequence of nucleotides of the mRNA transcripts; i.e., RNase H. Kits may further include instructions for using the kit according to a method of the invention.

In some embodiments, kits of the present invention may further include a control sample to be used as a reference point. For instance, a control sample included in the kit may include an mRNA sample comprising at least 90% of mRNA transcripts comprising the Cap 1 structure. In a specific embodiment, a control sample included in the kit comprises five, six, or seven ribonucleotides, as appropriate, wherein at least 90% of the ribonucleotides comprise the Cap 1 structure.

In some embodiments, materials and reagents for quantifying mRNA capping efficiency in an mRNA sample by enzymatic manipulation and chromatographic separation may be assembled together in a kit. For examples, such a kit may comprise agents for separating the first five, six, or seven nucleotides of the sequence of nucleotides of the mRNA transcripts comprising a cap structure on the column, chromatographic columns, and instructions for using the kit according to a method of the invention.

### Compositions

The present invention also provides purified mRNA compositions comprising in vitro synthesized mRNA comprising a Cap 1 structure. In some embodiments, the mRNA compositions comprises at least 80% of mRNA molecules comprising the Cap 1 structure. In some embodiments, the mRNA compositions comprises at least 85% of mRNA molecules comprising the Cap 1 structure. In some embodiments, the mRNA compositions comprises at least 90% of mRNA molecules comprising the Cap 1 structure. In some embodiments, the mRNA compositions comprises at least 95% of mRNA molecules comprising the Cap 1 structure. In some embodiments, the mRNA compositions comprises at least 96% of mRNA molecules comprising the Cap 1 structure. In some embodiments, the mRNA compositions comprises at least 97% of mRNA molecules comprising the Cap 1 structure. In some embodiments, the mRNA compositions comprises at least 98% of mRNA molecules comprising the Cap 1 structure. In some embodiments, the mRNA compositions comprises at least 99% of mRNA molecules comprising the Cap 1 structure. The mRNA molecules comprising the Cap 1 structure is prepared following the capping efficiency assay as described herein. In some embodiments, the percentage of the mRNA molecules comprising the Cap 1 structure is determined by the Capping efficiency assay of the present invention.

In some embodiments, the mRNA composition is encapsulated in a lipid nanoparticle. In some embodiments, the mRNA composition is formulated for in vivo delivery.

### EXAMPLE

### Example 1: Analysis of Capping Efficiency

This example demonstrates a method of quantifying capping efficiency in mRNA samples from two different in vitro transcription (IVT) reaction mixtures, referred to as “Sample 1” and “Sample 2”.

An oligonucleotide was designed and synthesized to be complementary to the mRNA transcripts to form an mRNA:DNA hybrid between the oligonucleotide and the second to fifth nucleotides of the sequence of nucleotides of the mRNA transcripts, as depicted in FIG. 3. The mRNA transcript comprised the following 5′ untranslated region (5′ UTR):

[SEQ ID NO: 2]
GGACAGAUCGCCUGGAGACGCCAUCCACGCUGUUUUGACCUCCAUAGAA
GACACCGGGACCGAUCCAGCCUCCGCGGCCGGGAACGGUGCAUUGGAAC
GCGGAUUCCCCGUGCCAAGAGUGACUCACCGUCCUUGACACG.

The sequence of the oligonucleotide was as follows: 5′-mUmCmCmAmGmGmCmGmAmUmCTGTCmC-3′ (SEQ ID NO: 1), wherein mU represents uridine with a 2′-O-methyl ribose; mC represents cytidine with a 2′-O-methyl ribose; mA represents adenine with a 2′-O-methyl ribose; mG represents guanidine with a 2′-O-methyl ribose; T represents deoxythymidine; G represents deoxyguanidine and C represents deoxycytidine and the underlined text represents DNA nucleotides.

Sample 1 and Sample 2 were processed in two separate reactions as follows. 55 pmol of mRNA sample comprising the mRNA transcripts was contacted with the oligonucleotide in a total initial assay volume of 22 μl and incubated for 10 minutes at 75° C., followed by 10 minutes at room temperature. The mRNA sample was then contacted with RNase H in the same reaction vessel to yield a total final assay volume of 52 μl. The reaction was allowed to proceed for 40 minutes at 37° C. The RNase H cleaved the mRNA in the region of the DNA:RNA hybrid, thereby releasing the first five 5′ nucleotides of the sequence of nucleotides of the mRNA transcripts.

Without any purification, the RNase H-cleaved mRNA sample was loaded onto a UHPLC-QTOF system (Agilent) with an Infinity Lab C18 2.1×100 mm column maintained at 50° C. to determine the portion of mRNA transcripts comprising a Cap 1 structure in the mRNA sample. Using a flow rate of 0.5 ml/min, a 10 minute 1-16% gradient from mobile phase A (100 mM HFIP, 8.6 mM trimethylamine, pH 8.3) to mobile phase B (100% methanol) was followed by 1.5 minutes of 50% mobile phase B. The eluate was directly analysed by QTOF spectrometry with 13 L/min 350° C. dry gas flow, 10 psi nebulizer pressure and a capillary voltage of 3750 V. Analysis was performed in the negative ion mode over a scan range of 400-3200 m/z.

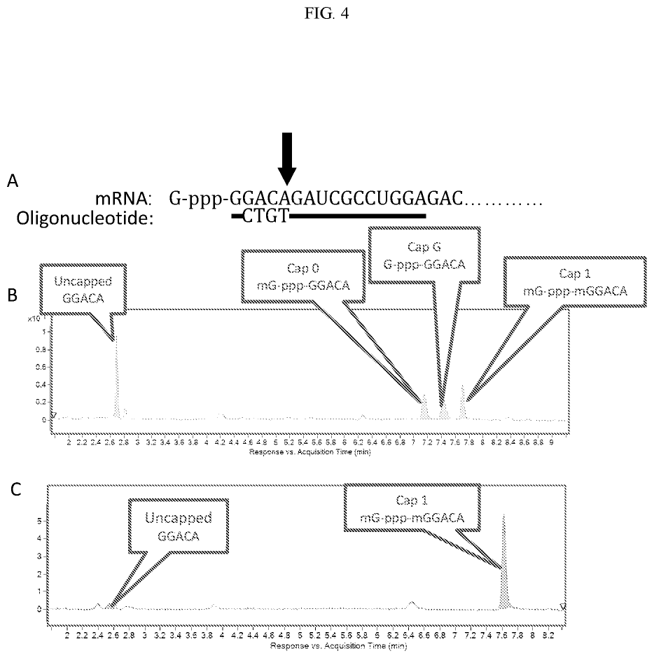

The resulting chromatograms for Sample 1 and Sample 2 are shown in FIG. 4B and FIG. 4C respectively. As shown in FIG. 4B, the chromatogram for Sample 1 revealed four discernable peaks. These could be assigned to uncapped nucleotides and nucleotides with Cap 0, Cap G and Cap 1, as indicated. As the portion of mRNA transcripts comprising the Cap 1 structure was less than 90%, Sample 1 was not further processed. As shown in FIG. 4C, the chromatogram for Sample 2 revealed a major peak corresponding to Cap 1. The relative abundance of Cap 1 to uncapped nucleotides was determined by automated integration of the relevant chromatographic peaks. The portion of mRNA transcripts comprising the Cap 1 structure was determined to be 95%, and therefore Sample 2 was taken for further processing.

1. A method of quantifying capping efficiency in an mRNA sample from an in vitro transcription (IVT) reaction mixture, wherein the method comprises:
(a) providing the mRNA sample, wherein the mRNA sample contains a plurality of mRNA transcripts comprising a sequence of nucleotides with or without a cap, wherein a first portion of the mRNA transcripts comprises a Cap 1 structure at the 5′ end of the first nucleotide of the sequence of nucleotides;
(b) contacting the mRNA sample with an oligonucleotide complementary to the mRNA transcripts to form an mRNA:DNA hybrid between the oligonucleotide and (i) the second to fifth nucleotides, (ii) the third to sixth nucleotides, or (iii) the fourth to seventh nucleotides of the sequence of nucleotides of the mRNA transcripts;
(c) contacting the sample obtained in step (b) with RNase H to release (i) the first five nucleotides, (ii) the first six nucleotides, or (iii) the first seven nucleotides, respectively, of the sequence of nucleotides of the mRNA transcripts;
(d) analyzing the sample obtained in step (c) to determine the first portion of mRNA transcripts comprising the Cap 1 structure in the mRNA sample,
wherein steps (a) to (c) proceed subsequent to one another in the same assay vessel.

2. The method according to claim 1, wherein steps (b) to (d) are performed by an automated system.

3. The method according to claim 1, wherein the analysis further determines the methylation status of the cap.

4. The method according to claim 1, wherein the cap is added enzymatically post transcription.

5. The method according to claim 1, wherein the cap is added to the mRNA co-transcriptionally.

6. The method according to claim 1, wherein the oligonucleotide is represented by the following formula: 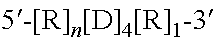
wherein each R is an RNA nucleotide, each D is a DNA nucleotide, wherein n is between 10 and 20, the oligonucleotide has a GC content of about 40% to about 60%, and the mRNA:DNA hybrid has a melting temperature between about 50° C. and about 60° C.

7. The oligonucleotide of claim 6, wherein each of the RNA nucleotides comprises a 2′-O-methyl ribose.

8. The method according to claim 1, wherein:
(i) the method requires an input of not more than 100 pmol of in vitro transcribed mRNA in the mRNA sample provided in step (a);
(ii) the method requires a total assay volume of not more than 100 μl; and/or
(iii) steps (b) to (c) are performed in 90 minutes or less.

9-10. (canceled)

11. The method according to claim 1, wherein the analysis in step (d) comprises high performance liquid chromatography (HPLC) or ultra-high performance liquid chromatography (UHPLC) to separate the first five, six, or seven nucleotides of the sequence of nucleotides of the mRNA transcripts released in step (c) to determine the presence or absence of the cap.

12. (canceled)

13. The method according to claim 11, wherein the analysis in step (d) further comprises mass spectrometry (MS) or liquid chromatography-mass spectrometry (LC-MS) to identify the first five, six, or seven nucleotides of the sequence of nucleotides of the mRNA transcripts released in step (c) that comprise the Cap 1 structure.

14. (canceled)

15. The method according to claim 13, wherein the analysis of the sample according to step (d) by MS comprises analysis over a scan range of 200-6000 m/z.

16. The method of claim 1, wherein the mRNA sample provided in step (a) comprises a second portion of mRNA transcripts comprising a Cap 0 structure.

17. The method of claim 1, wherein the mRNA sample provided in step (a) comprises a third portion of mRNA transcripts comprising a Cap G structure.

18. The method according to claim 1, wherein the first portion of mRNA transcripts comprising the Cap 1 structure is at least 90% for the IVT reaction mixture to be processed further.

19. The method according to claim 1, wherein the second portion of mRNA transcripts comprising the Cap 0 structure is not more than 10% for the IVT reaction mixture to be processed further.

20. The method according to claim 1, wherein the third portion of mRNA transcripts comprising the Cap G structure is not more than 10% for the IVT reaction mixture to be processed further.

21. The method according to claim 18, wherein the further processing includes purification and/or encapsulation of the mRNA transcripts from the IVT reaction mixture.

22. A method of manufacturing a therapeutic mRNA, wherein said method comprises:
(i) synthesizing the therapeutic mRNA using an in vitro transcription (IVT) reaction mixture;
(ii) analyzing an mRNA sample from the in vitro transcription (IVT) reaction mixture using the method of claim 1; and
(iii) further processing the mRNA, if the first portion of mRNA transcripts comprising the Cap 1 structure in the mRNA is at least 90%.

23. The method of claim 22, wherein further processing comprises purification of the therapeutic mRNA synthesized in step (i) and/or formulating the therapeutic mRNA synthesized in step (i).

24. (canceled)

25. The method of claim 23, wherein formulation comprises encapsulating the mRNA in a lipid nanoparticle.
